(19) United States
(12) Patent Application Publication
(10) Pub. No.: US 2025/0279210 A1
(43) Pub. Date: Sep. 4, 2025

(54) SYSTEMS AND METHODS FOR NUCLEAR MAGNETIC RESONANCE BASED METABOLOMICS

(71) Applicant: lifespin GmbH, Regensburg (DE)

(72) Inventors: Ali TINAZLI, Kelkheim (DE); Roland GEYER, Rohr in NB (DE); Michael RETTIG, Regensburg (DE); Steffen HEELEMANN, Bach an der Donau (DE); Johannes WITTMANN, Regensburg (DE); Tobias AMEISMEIER, Köfering (DE); Armin REGLER, Bavaria (DE)

(21) Appl. No.: 19/052,089

(22) Filed: Feb. 12, 2025

Related U.S. Application Data
(63) Continuation of application No. PCT/EP2023/072724, filed on Aug. 17, 2023.
(60) Provisional application No. 63/371,826, filed on Aug. 18, 2022.

(51) Int. Cl. G16H 50/30; G01N 24/08; G06N 20/00

(52) U.S. Cl. CPC G16H 50/30; G01N 24/08; G06N 20/00; G01N 2800/7028

(57) ABSTRACT

Described herein are methods and systems for processing a biological sample of a subject to determine whether the subject has or is at an increased risk of developing a disease or disorder. Methods of the present disclosure may be performed using a trained machine learning algorithm to predict disease or risk of disease, identify disease, or monitor disease. Methods may be performed using a nuclear magnetic resonance (NMR) spectrum of a subject or a metabolome of the subject. Methods may predict, identify, or monitor diseases such as cancers, metabolic diseases or disorders, inflammatory diseases or disorders, or neurological diseases or disorders. Methods may be performed using a cloud computing system. Methods may provide a report to the subject indicating whether the subject has or is at an increased risk of developing a disease or disorder.

### CROSS-REFERENCE

This application is a continuation application of International Patent Application No. PCT/EP2023/072724, filed on Aug. 17, 2023, which claims the benefit of U.S. Provisional Application No. 63/371,826, filed on Aug. 18, 2022, each of which application is incorporated herein by reference.

### BACKGROUND

Many diseases may be linked to metabolism and metabolic disorders thereof. Unfortunately, the state of affairs for assessing whether a subject has or is at an increased risk of developing a disease or disorder is deficient in many ways. First, some methods may require invasive procedures to obtain a sample (e.g., human sample) to determine the disease or disorder. Also, some methods may require a substantial amount or a repeated amount of the sample thereby increasing invasiveness of the procedure. Further, some solutions may require substantial time to process the sample to determine the disease or disorder thereby delaying treatment of the disease. Additionally, some methods may be prohibitively costly thereby denying timely assessment and treatment of the disease. Some methods may only provide subjective assessment of the disease instead of reliably and digitally quantifying parameters of the disease. As such some methods may not be able to predict, with a high level of confidence, whether the subject has or is at an increased risk of developing the disease.

### SUMMARY

The present disclosure provides methods for processing and/or analyzing a biological sample to determine whether a subject has or is at an increased risk of developing a disease or disorder, such as cancer. Methods of the present disclosure May 1) use noninvasive procedures to collect and process a sample (e.g., human sample), 2) require inconsequential amounts of the sample, 3) provide real-time or near real-time analysis of the sample to assess disease or disorder, 4) cost less to assess the disease or disorder as compared to other methods, 5) provide digital, quantitative measurement of parameters indicative of the disease or disorder, or 6) predict the disease or disorder with a high level of confidence as compared to other methods. Doing so may provide unique and immediate clinical insights into the disease or disorder.

The present disclosure provides methods for determining whether a subject has or is at an increased risk of developing a disease (e.g., cancer) or disorder based at least on using a nuclear magnetic resonance (NMR) instrument to generate an NMR spectrum or a metabolome derived from the NMR spectrum. Methods described herein may process a biological sample of the subject to determine whether the subject has or is at an increased risk of developing a disease or disorder. Methods described herein may identify that the subject has or is suspected of having a disease or disorder. Methods described herein may monitor the subject who has or is suspected of having a disease or disorder. Methods described herein may be performed using machine learning to determine or predict that the subject has or is at an increased risk of developing a disease or disorder. Methods described herein may be performed using a cloud computing system to determine that the subject has or is at an increased risk of developing a disease or disorder. Methods described herein may receive a report or provide a report to the subject indicative of the subject having or being at increased risk of developing a disease or disorder with a high level of confidence. In addition, methods of the present disclosure are not limited to NMR spectroscopy and may be employed by other approaches, such as, for example, mass spectroscopy. For example, mass spectroscopy may be used to obtain a metabolic profile of a sample of a subject for use in determine whether the subject has or is at an increased risk of developing a disease or disorder.

Methods described herein may use a NMR spectrum or a metabolome derived from the NMR spectrum. Additionally, methods described herein may use a metabolome derived from other than an NMR spectrum. For example, a metabolome may be derived from liquid chromatography, gas chromatography, mass spectrometry, or combinations thereof and processed by methods disclosed herein to determine whether a subject has or is at an increased risk of developing a disease or disorder. Further, methods described herein are not limited to their use in metabolomics. For example, other types of omics may be derived and used to complement methods described herein to determine whether a subject has or is at an increased risk of developing a disease or disorder. Other types of complementary omics may include genomics, transcriptomics, or proteomics. In this sense, methods described herein may provide an integrated or multi-omics approach to determining whether a subject has or is at an increased risk of developing a disease with a high level of confidence.

In an aspect, disclosed herein is a method of processing a biological sample of a subject, of the biological sample of the subject; (b) computer processing the NMR spectrum or a metabolome derived from the NMR spectrum to determine that the subject has or is at an increased risk of developing a disease, wherein the NMR spectrum or the metabolome corresponds to a plurality of metabolites; and (c) electronically outputting a report indicative of the subject having or being at the increased risk of developing the disease.

In some embodiments, (b) or (c) is performed using a cloud computing system. In some embodiments, (b) and (c) are performed using a cloud computing system.

In some embodiments, (b) is performed using a trained machine learning algorithm. In some embodiments, the trained machine learning algorithm is trained using at least 1000 biological samples. In some embodiments, (b) and (c) are performed using one or more trained machine learning algorithms.

In some embodiments, the metabolome is a full metabolome. In some embodiments, the full metabolome is a full metabolome to a detection limit of the NMR instrument. In some embodiments, the plurality of metabolites comprises 3 or more metabolites. In some embodiments, the plurality of metabolites comprises 4 or more metabolites. In some embodiments, the plurality of metabolites comprises 5 or more metabolites. In some embodiments, the plurality of metabolites comprises 10 or more metabolites. In some embodiments, the plurality of metabolites comprises 50 or more metabolites.

In some embodiments, the disease is a cancer. In some embodiments, the cancer comprises lung cancer, thyroid cancer, colorectal cancer, breast cancer, prostate cancer, rectal cancer, chronic lymphatic leukemia, pancreatic cancer, or ovarian cancer. In some embodiments, the disease is a metabolic disease. In some embodiments, the metabolic disease comprises kidney disease or liver disease. In some embodiments, the disease is an inflammatory disease. In some embodiments, the inflammatory disease comprises rheumatoid arthritis or ulcerative colitis. In some embodiments, the disease is a neurological disease. In some embodiments, the neurological disease comprises multiple sclerosis, Parkinson's disease, or Alzheimer's disease.

In some embodiments, the trained machine learning algorithm comprises use of one or more artificial neural networks, random forests, support vector machines, linear regressions, nonlinear regressions, or clusterings. In some embodiments, the trained machine learning algorithm comprises use of the one or more artificial neural networks. In some embodiments, the one or more artificial neural networks comprises use of one or more self-organizing maps.

In some embodiments, the biological sample has not been purified. In some embodiments, the biological sample comprises bile acids, blood (e.g., arterial, capillary, umbilical cord, venous, whole), bronchoalveolar lavage fluid, cell extracts, cerebrospinal fluid, exhaled breath condensates (EBC), plasma (e.g., citrate, EDTA, hirudin, lithium heparin, sodium fluoride), saliva, serum, sperm, stool, sweat, tear fluid, tracheal wash (TW), or urine.

In some embodiments, the computer processing comprises use of a database comprising at least about 100,000 NMR spectra related to other metabolomes. In some embodiments, the computer processing comprises determining a deviation of the NMR spectrum or the metabolome from a healthy set of information stored in the database.

In some embodiments, a time from (a) to (c) is less than about 60 minutes. In some embodiments, a time from (a) to (c) is less than about 30 minutes.

In some embodiments, the metabolome comprises information related to a number of at least about 50 metabolites. In some embodiments, the metabolome comprises information related to a number of at least about 100 metabolites. In some embodiments, the metabolome comprises information related to a number of at least about 500 metabolites. In some embodiments, the metabolome is not deconvoluted.

In some embodiments, the report indicates that the subject has or is at an increased risk of developing the disease at an accuracy of least about 60%, 70%, 80%, 85%, 90%, 95%, or greater. In some embodiments, the report indicates that the subject has or is at an increased risk of developing the disease at a sensitivity of least about 60%, 70%, 80%, 85%, 90%, 95%, or greater. In some embodiments, the report indicates that the subject has or is at an increased risk of developing the disease at a specificity of least about 60%, 70%, 80%, 85%, 90%, 95%, or greater.

In some embodiments, the biological sample is not destroyed In some embodiments, the biological sample is derived from an animal. In some embodiments, the biological sample is derived from a human.

In some embodiments, the report comprises one or more diagnostics. In some embodiments, the report comprises a lipid profile.

In some embodiments, the computer processing comprises deconvoluting the spectrum or the metabolome to determine a presence or absence of the plurality of metabolites. In some embodiments, the plurality of metabolites are determined quantitatively.

In another aspect, disclosed herein is a method of processing a biological sample of a subject, comprising: (a) using a nuclear magnetic resonance (NMR) instrument to generate an NMR spectrum of the biological sample of the subject; (b) computer processing the NMR spectrum to identify a metabolome of the biological sample; (c) computer processing the metabolome to determine that the subject has a disease at a specificity of at least 80%; and (d) electronically outputting a report indicative as to whether the subject has or is at the increased risk of developing the disease.

In some embodiments, the (b), (c) or (d) is performed using a cloud computing system. In some embodiments, the (b), (c) and (d) are performed using a cloud computing system. In some embodiments, the (b) or (c) is performed using a trained machine learning algorithm. In some embodiments, the trained machine learning algorithm is trained using at least 1000 biological samples. In some embodiments, the (b), (c) or (d) is performed using one or more trained machine learning algorithms.

In some embodiments, the metabolome is a full metabolome. In some embodiments, the full metabolome is a full metabolome to a detection limit of the NMR instrument. In some embodiments, the metabolome corresponds to a plurality of metabolites. In some embodiments, the plurality of metabolites comprises 3 or more metabolites. In some embodiments, the plurality of metabolites comprises 4 or more metabolites. In some embodiments, In some embodiments, the plurality of metabolites comprises 5 or more metabolites. In some embodiments, the plurality of metabolites comprises 10 or more metabolites. In some embodiments, the plurality of metabolites comprises 50 or more metabolites.

In some embodiments, the disease is cancer. In some embodiments, the cancer comprises lung cancer, thyroid cancer, colorectal cancer, breast cancer, prostate cancer, rectal cancer, chronic lymphatic leukemia, pancreatic cancer, or ovarian cancer. In some embodiments, the disease is a metabolic disease. In some embodiments, the metabolic disease comprises kidney disease or liver disease. In some embodiments, the disease is an inflammatory disease. In some embodiments, the inflammatory disease comprises rheumatoid arthritis or ulcerative colitis. In some embodiments, the disease is a neurological disease. In some embodiments, the neurological disease comprises multiple sclerosis, Parkinson's disease, or Alzheimer's disease.

In some embodiments, the trained machine learning algorithm comprises use of one or more artificial neural networks, random forests, support vector machines, linear regressions, nonlinear regressions, or clusterings. In some embodiments, the trained machine learning algorithm comprises use of the one or more artificial neural networks. In some embodiments, the one or more artificial neural networks comprises use of one or more self-organizing maps.

In some embodiments, the biological sample has not been purified. In some embodiments, the biological sample comprises bile acids, blood (e.g., arterial, capillary, umbilical cord, venous, whole), bronchoalveolar lavage fluid, cell extracts, cerebrospinal fluid, exhaled breath condensates (EBC), plasma (e.g., citrate, EDTA, hirudin, lithium heparin, sodium fluoride), saliva, serum, sperm, stool, sweat, tear fluid, tracheal wash (TW), or urine.

In some embodiments, the computer processing comprises use of a database comprising at least about 100,000 NMR spectra related to other metabolomes. In some embodiments, the computer processing comprises determining a deviation of the NMR spectrum or the metabolome from a healthy set of information stored in the database.

In some embodiments, a time from (a) to (d) is less than about 60 minutes. In some embodiments, a time from (a) to (d) is less than about 30 minutes.

In some embodiments, the metabolome comprises information related to a number of at least about 50 metabolites. In some embodiments, the metabolome comprises information related to a number of at least about 100 metabolites. In some embodiments, the metabolome comprises information related to a number of at least about 500 metabolites. In some embodiments, the metabolome is not deconvoluted.

In some embodiments, the report has an accuracy, selectivity, or specificity of at least about 85%, 90%, 95%, or greater.

In some embodiments, the biological sample is not destroyed. In some embodiments, the biological sample is derived from a human. In some embodiments, the biological sample is derived from an animal.

In some embodiments, the report comprises one or more diagnostics. In some embodiments, the report comprises lipid profiling.

In some embodiments, the computer processing comprises deconvoluting the spectrum or the metabolome to determine a presence or absence of the plurality of metabolites. In some embodiments, the plurality of metabolites are determined quantitatively.

In another aspect, disclosed herein is a method of processing a biological sample of a subject, of the biological sample of the subject; (b) transmitting the NMR spectrum or a metabolome derived from the NMR spectrum to a cloud computing system, which cloud computing system processes the NMR spectrum or the metabolome to determine that the subject has or is at an increased risk of having a disease; and (c) receiving an electronic report indicative of the subject having or being at the increased risk of developing the disease.

In some embodiments, the (b) or (c) is performed using a cloud computing system. In some embodiments, the (b) and (c) are performed using a cloud computing system. In some embodiments, the cloud computing system processes the NMR spectrum using a trained machine learning algorithm. In some embodiments, the trained machine learning algorithm is trained using at least 1000 biological samples. In some embodiments, the cloud computing system processes the NMR spectrum using one or more trained machine learning algorithms.

In some embodiments, the metabolome is a full metabolome. In some embodiments, the full metabolome is a full metabolome to a detection limit of the NMR instrument. In some embodiments, the metabolome corresponds to a plurality of metabolites. In some embodiments, the plurality of metabolites comprises 3 or more metabolites. In some embodiments, the plurality of metabolites comprises 4 or more metabolites. In some embodiments, the plurality of metabolites comprises 5 or more metabolites. In some embodiments, the plurality of metabolites comprises 10 or more metabolites. In some embodiments, the plurality of metabolites comprises 50 or more metabolites.

In some embodiments, the disease is a cancer. In some embodiments, the cancer comprises lung cancer, thyroid cancer, colorectal cancer, breast cancer, prostate cancer, rectal cancer, chronic lymphatic leukemia, pancreatic cancer, or ovarian cancer. In some embodiments, the disease is a metabolic disease. In some embodiments, the metabolic disease comprises kidney disease or liver disease. In some embodiments, the disease is an inflammatory disease. In some embodiments, In some embodiments, the inflammatory disease comprises rheumatoid arthritis or ulcerative colitis. In some embodiments, the disease is a neurological disease. In some embodiments, the neurological disease comprises multiple sclerosis, Parkinson's disease, or Alzheimer's disease.

In some embodiments, the trained machine learning algorithm comprises use of one or more artificial neural networks, random forests, support vector machines, linear regressions, nonlinear regressions, or clusterings. In some embodiments, the trained machine learning algorithm comprises use of the one or more artificial neural networks. In some embodiments, the one or more artificial neural networks comprises use of one or more self-organizing maps.

In some embodiments, the biological sample has not been purified. In some embodiments, the biological sample comprises bile acids, blood (e.g., arterial, capillary, umbilical cord, venous, whole), bronchoalveolar lavage fluid, cell extracts, cerebrospinal fluid, exhaled breath condensates (EBC), plasma (e.g., citrate, EDTA, hirudin, lithium heparin, sodium fluoride), saliva, serum, sperm, stool, sweat, tear fluid, tracheal wash (TW), or urine.

In some embodiments, the cloud computing system processing comprises use of a database comprising at least about 100,000 NMR spectra related to other metabolomes. In some embodiments, the cloud computing system processing comprises determining a deviation of the NMR spectrum or the metabolome from a healthy set of information stored in the database.

In some embodiments, a time from (a) to (c) is less than about 60 minutes. In some embodiments, a time from (a) to (c) is less than about 30 minutes.

In some embodiments, the metabolome comprises information related to a number of at least about 50 metabolites. In some embodiments, the metabolome comprises information related to a number of at least about 100 metabolites. In some embodiments, the metabolome comprises information related to a number of at least about 500 metabolites. In some embodiments, the metabolome is not deconvoluted. In some embodiments, the report has an accuracy, selectivity, or specificity of at least about 80%, 85%, 90%, 95%, or greater.

In some embodiments, the biological sample is not destroyed. In some embodiments, the biological sample is derived from a human. In some embodiments, the biological sample is derived from an animal.

In some embodiments, the report comprises one or more diagnostics. In some embodiments, the report comprises lipid profiling.

In some embodiments, the cloud computing system processing comprises deconvoluting the spectrum or the metabolome to determine a presence or absence of the plurality of metabolites. In some embodiments, the plurality of metabolites are determined quantitatively.

In another aspect, disclosed herein is a method of processing a biological sample of a subject, comprising: (a) receiving a nuclear magnetic resonance (NMR) spectrum or a metabolome derived from the NMR spectrum through a cloud computing system, wherein the NMR spectrum is generated from the biological sample of the subject, and wherein the cloud computing system processes the NMR spectrum or the metabolome to determine that the subject has or is at an increased risk of having a disease; and (b) transmitting an electronic report indicative of the subject having or being at the increased risk of developing the disease.

In some embodiments, the (b) is performed using a cloud computing system. In some embodiments, the (a) or (b) is performed using a trained machine learning algorithm. In some embodiments, the trained machine learning algorithm is trained using at least 1000 biological samples. In some embodiments, the (a) and (b) are performed using one or more trained machine learning algorithms.

In some embodiments, the metabolome is a full metabolome. In some embodiments, the full metabolome is a full metabolome to a detection limit of the NMR instrument. In some embodiments, the metabolome corresponds to a plurality of metabolites. In some embodiments, the plurality of metabolites comprises 3 or more metabolites. In some embodiments, the plurality of metabolites comprises 4 or more metabolites. In some embodiments, the plurality of metabolites comprises 5 or more metabolites. In some embodiments, the plurality of metabolites comprises 10 or more metabolites. In some embodiments, the plurality of metabolites comprises 50 or more metabolites.

In some embodiments, the disease is a cancer. In some embodiments, the cancer comprises lung cancer, thyroid cancer, colorectal cancer, breast cancer, prostate cancer, rectal cancer, chronic lymphatic leukemia, pancreatic cancer, or ovarian cancer. In some embodiments, the disease is a metabolic disease. In some embodiments, the metabolic disease comprises kidney disease or liver disease. In some embodiments, the disease is an inflammatory disease. In some embodiments, the inflammatory disease comprises rheumatoid arthritis or ulcerative colitis. In some embodiments, the disease is a neurological disease. In some embodiments, the neurological disease comprises multiple sclerosis, Parkinson's disease, or Alzheimer's disease.

In some embodiments, the trained machine learning algorithm comprises use of a neural network. In some embodiments, the neural network comprises one or more of random forests, support vector machines, linear regression, nonlinear regression, clustering algorithms, or self-organizing maps. In some embodiments, the neural network comprises a self-organizing map.

In some embodiments, the biological sample has not been purified. In some embodiments, the biological sample comprises bile acids, blood (e.g., arterial, capillary, umbilical cord, venous, whole), bronchoalveolar lavage fluid, cell extracts, cerebrospinal fluid, exhaled breath condensates (EBC), plasma (e.g., citrate, EDTA, hirudin, lithium heparin, sodium fluoride), saliva, serum, sperm, stool, sweat, tear fluid, tracheal wash (TW), or urine.

In some embodiments, the cloud computing system comprises use of a database comprising at least about 100,000 NMR spectra related to other metabolomes. In some embodiments, the cloud computing system comprises determining a deviation of the NMR spectrum or the metabolome from a healthy set of information stored in the database.

In some embodiments, a time from (a) to (b) is less than about 60 minutes. In some embodiments, a time from (a) to (b) is less than about 30 minutes.

In some embodiments, the metabolome comprises information related to a number of at least about 50 metabolites. In some embodiments, the metabolome comprises information related to a number of at least about 100 metabolites. In some embodiments, the metabolome comprises information related to a number of at least about 500 metabolites. In some embodiments, the metabolome is not deconvoluted.

In some embodiments, the report has an accuracy, selectivity, or specificity of at least about 80%, 85%, 90%, 95%, or greater.

In some embodiments, the biological sample is not destroyed. In some embodiments, the biological sample is derived from a human. In some embodiments, the biological sample is derived from an animal.

In some embodiments, the report comprises one or more diagnostics. In some embodiments, the report comprises lipid profiling.

In some embodiments, the cloud computing system comprises deconvoluting the spectrum or the metabolome to determine a presence or absence of the plurality of metabolites. In some embodiments, the plurality of metabolites are determined quantitatively.

In another aspect, disclosed herein is a method, comprising: (a) receiving a plurality of nuclear magnetic resonance (NMR) spectra, wherein the plurality of NMR spectra are of a plurality of training samples from a plurality of subjects having been identified as having at least one disease; and (b) using the plurality of NMR spectra or a plurality of metabolomes derived from the plurality of NMR spectra to train a machine learning algorithm to identify at least one feature of the plurality of NMR spectra or the plurality of metabolomes, to yield a trained machine learning algorithm that is configured to (i) take as input an NMR spectrum of a biological sample of a subject or a metabolome derived from the NMR spectrum and (ii) process the NMR spectrum or the metabolome to determine that the subject has or is at an increased risk of having a disease of the at least one disease.

In some embodiments, the (a) or (b) is performed using a cloud computing system. In some embodiments, the (a) and (b) are performed using a cloud computing system. In some embodiments, the NMR spectra is generated by an NMR instrument. In some embodiments, the trained machine learning algorithm is trained using at least 1000 training samples.

In some embodiments, the metabolome is a full metabolome. In some embodiments, the full metabolome is a full metabolome to a detection limit of the NMR instrument. In some embodiments, the metabolome corresponds to a plurality of metabolites. In some embodiments, the plurality of metabolites comprises 3 or more metabolites. In some embodiments, the plurality of metabolites comprises 4 or more metabolites. In some embodiments, the plurality of metabolites comprises 5 or more metabolites. In some embodiments, the plurality of metabolites comprises 10 or more metabolites. In some embodiments, the plurality of metabolites comprises 50 or more metabolites.

In some embodiments, the disease is a cancer. In some embodiments, the cancer comprises lung cancer, thyroid cancer, colorectal cancer, breast cancer, prostate cancer, rectal cancer, chronic lymphatic leukemia, pancreatic cancer, or ovarian cancer. In some embodiments, the disease is a metabolic disease. In some embodiments, the metabolic disease comprises kidney disease or liver disease. In some embodiments, the disease is an inflammatory disease. In some embodiments, the inflammatory disease comprises rheumatoid arthritis or ulcerative colitis. In some embodiments, the disease is a neurological disease. In some embodiments, the neurological disease comprises multiple sclerosis, Parkinson's disease, or Alzheimer's disease.

In some embodiments, the trained machine learning algorithm comprises use of one or more artificial neural networks, random forests, support vector machines, linear regressions, nonlinear regressions, or clusterings. In some embodiments, the trained machine learning algorithm comprises use of the one or more artificial neural networks. In some embodiments, the one or more artificial neural networks comprises use of one or more self-organizing maps.

In some embodiments, the biological sample has not been purified. In some embodiments, the biological sample comprises bile acids, blood (e.g., arterial, capillary, umbilical cord, venous, whole), bronchoalveolar lavage fluid, cell extracts, cerebrospinal fluid, exhaled breath condensates (EBC), plasma (e.g., citrate, EDTA, hirudin, lithium heparin, sodium fluoride), saliva, serum, sperm, stool, sweat, tear fluid, tracheal wash (TW), or urine.

In some embodiments, the trained machine learning algorithm is trained using the plurality of NMR spectra comprising at least about 100,000 NMR spectra related to other metabolomes. In some embodiments, the trained machine learning algorithm determines a deviation of the NMR spectrum or the metabolome from a healthy set of information.

In some embodiments, a time from (b) (i) to (b) (ii) is less than about 60 minutes. In some embodiments, a time from (b) (i) to (b) (ii) is less than about 30 minutes.

In some embodiments, the metabolome comprises information related to a number of at least about 50 metabolites. In some embodiments, the metabolome comprises information related to a number of at least about 100 metabolites. In some embodiments, the metabolome comprises information related to a number of at least about 500 metabolites. In some embodiments, the metabolome is not deconvoluted.

In some embodiments, the trained machine learning algorithm has an accuracy, selectivity, or specificity of at least about 80%, 85%, 90%, 95%, or greater.

In some embodiments, the biological sample is not destroyed. In some embodiments, the biological sample is derived from a human. In some embodiments, the biological sample is derived from an animal. In some embodiments,

In some embodiments, the method further comprises electronically outputting a report indicative of the subject having or being at the increased risk of having the disease. In some embodiments, the report comprises lipid profiling.

In some embodiments, the receiving in (a) further comprises deconvoluting the spectrum or the metabolome to determine a presence or absence of a plurality of metabolites. In some embodiments, the plurality of metabolites are determined quantitatively.

In some embodiments, the features in (b) comprise at least about 2, 3, 4, 5, or more features.

In another aspect, disclosed herein is a method for monitoring a health or physiological condition of a subject, comprising: (a) obtaining a first NMR or metabolome spectrum of the subject at a first time point; (b) obtaining a second NMR or metabolome spectrum of the subject at a second time point; and (c) computer processing the first NMR or metabolome spectrum and second first NMR or metabolome spectrum, thereby monitoring the health or physiological condition of the subject.

In some embodiments, the subject has or is suspected of having a disease, and wherein monitoring the health or physiological condition of the subject comprises monitoring a progression or regression of the disease.

In some embodiments, the disease is a cancer comprising lung cancer, thyroid cancer, colorectal cancer, breast cancer, prostate cancer, rectal cancer, chronic lymphatic leukemia, pancreatic cancer, or ovarian cancer. In some embodiments, the disease is a metabolic disease comprising kidney disease or liver disease. In some embodiments, the disease is an inflammatory disease comprising rheumatoid arthritis or ulcerative colitis. In some embodiments, the disease is a neurological disease comprising multiple sclerosis, Parkinson's disease, or Alzheimer's disease.

In some embodiments, the second time point occurs about 1 week, 1 month, 1 year, or more after the first time point.

In some embodiments, the first or second NMR spectrum or the first or second metabolome spectrum are received by processing a plurality of biological samples according to the methods described herein.

In another aspect, disclosed herein is a method for identifying a health or physiological condition of a subject, comprising: (a) receiving, from a digital computer over a computer network, an NMR or metabolome spectrum of the subject; (b) computer processing the NMR or metabolome spectrum to identify the health or physiological condition of the subject; and (c) transmitting, to the digital computer over the computer network, an electronic report indicative of the health or physiological condition of the subject identified in (b).

In some embodiments, the subject has or is suspected of having a disease, and wherein identifying the health or physiological condition of the subject comprises identifying the disease.

In some embodiments, the disease is a cancer comprising lung cancer, thyroid cancer, colorectal cancer, breast cancer, prostate cancer, rectal cancer, chronic lymphatic leukemia, pancreatic cancer, or ovarian cancer. In some embodiments, the disease is a metabolic disease comprising kidney disease or liver disease. In some embodiments, the disease is an inflammatory disease comprising rheumatoid arthritis or ulcerative colitis. In some embodiments, the disease is a neurological disease comprising multiple sclerosis, Parkinson's disease, or Alzheimer's disease.

In some embodiments, the NMR or metabolome spectrum of the subject are received by processing a plurality of biological samples according to the methods described herein.

In another aspect, disclosed herein is a method for identifying a health or physiological condition of a subject, comprising: (a) processing a biological sample of the subject to generate an NMR or metabolome spectrum of the subject; (b) transmitting, over a computer network, the NMR or metabolome spectrum of the subject to a computer system that (i) receives the NMR or metabolome spectrum of the subject and (ii) processes the NMR or metabolome spectrum of the subject to identify the health or physiological condition of the subject; and (c) receiving, over the computer network, an electronic report indicative of the health or physiological condition of the subject identified in (b).

In some embodiments, the subject has or is suspected of having a disease, and wherein identifying the health or physiological condition of the subject comprises identifying the disease.

In some embodiments, the disease is a cancer comprising lung cancer, thyroid cancer, colorectal cancer, breast cancer, prostate cancer, rectal cancer, chronic lymphatic leukemia, pancreatic cancer, or ovarian cancer. In some embodiments, the disease is a metabolic disease comprising kidney disease or liver disease. In some embodiments, the disease is an inflammatory disease comprising rheumatoid arthritis or ulcerative colitis. In some embodiments, the disease is a neurological disease comprising multiple sclerosis, Parkinson's disease, or Alzheimer's disease.

In some embodiments, the NMR or metabolome spectrum of the subject are received by processing a plurality of biological samples according to the methods described herein.

In another aspect, disclosed herein is a method of processing a biological sample of a subject, comprising: (a) using a nuclear magnetic resonance (NMR) instrument to generate an NMR spectrum of the biological sample of the subject; (b) computer processing the NMR spectrum or a metabolome derived from the NMR spectrum to determine that the subject does not have or is not at an increased risk of developing a disease, wherein the NMR spectrum or the metabolome corresponds to a plurality of metabolites; and (c) electronically outputting a report indicative of the subject not having or not being at the increased risk of developing the disease.

In some embodiments, the (b) or (c) is performed using a cloud computing system. In some embodiments, the (b) and (c) are performed using a cloud computing system. In some embodiments, the (b) is performed using a trained machine learning algorithm. In some embodiments, the trained machine learning algorithm is trained using at least 1000 biological samples. In some embodiments, the (b) and (c) are performed using one or more trained machine learning algorithms.

In some embodiments, the metabolome is a full metabolome. In some embodiments, the full metabolome is a full metabolome to a detection limit of the NMR instrument. In some embodiments, the plurality of metabolites comprises 3 or more metabolites. In some embodiments, the plurality of metabolites comprises 4 or more metabolites. In some embodiments, the plurality of metabolites comprises 5 or more metabolites. In some embodiments, the plurality of metabolites comprises 10 or more metabolites. In some embodiments, the plurality of metabolites comprises 50 or more metabolites.

In some embodiments, the disease is a cancer. In some embodiments, the cancer comprises lung cancer, thyroid cancer, colorectal cancer, breast cancer, prostate cancer, rectal cancer, chronic lymphatic leukemia, pancreatic cancer, or ovarian cancer. In some embodiments, the disease is a metabolic disease. In some embodiments, the metabolic disease comprises kidney disease or liver disease. In some embodiments, the disease is an inflammatory disease. In some embodiments, the inflammatory disease comprises rheumatoid arthritis or ulcerative colitis. In some embodiments, the disease is a neurological disease. In some embodiments, the neurological disease comprises multiple sclerosis, Parkinson's disease, or Alzheimer's disease.

In some embodiments, the trained machine learning algorithm comprises use of one or more artificial neural networks, random forests, support vector machines, linear regressions, nonlinear regressions, or clusterings. In some embodiments, the trained machine learning algorithm comprises use of the one or more artificial neural networks. In some embodiments, the one or more artificial neural networks comprises use of one or more self-organizing maps.

In some embodiments, the biological sample has not been purified. In some embodiments, the biological sample comprises bile acids, blood (e.g., arterial, capillary, umbilical cord, venous, whole), bronchoalveolar lavage fluid, cell extracts, cerebrospinal fluid, exhaled breath condensates (EBC), plasma (e.g., citrate, EDTA, hirudin, lithium heparin, sodium fluoride), saliva, serum, sperm, stool, sweat, tear fluid, tracheal wash (TW), or urine.

In some embodiments, the computer processing comprises use of a database comprising at least about 100,000 NMR spectra related to other metabolomes. In some embodiments, the computer processing comprises determining a deviation of the NMR spectrum or the metabolome from a healthy set of information stored in the database.

In some embodiments, a time from (a) to (c) is less than about 60 minutes. In some embodiments, a time from (a) to (c) is less than about 30 minutes.

In some embodiments, metabolome comprises information related to a number of at least about 50 metabolites. In some embodiments, the metabolome comprises information related to a number of at least about 100 metabolites. In some embodiments, the metabolome comprises information related to a number of at least about 500 metabolites. In some embodiments, the metabolome is not deconvoluted.

In some embodiments, the report has an accuracy, selectivity, or specificity of at least about 80%, 85%, 90%, 95%, or greater.

In some embodiments, the biological sample is not destroyed. In some embodiments, the biological sample is derived from a human. In some embodiments, the biological sample is derived from an animal.

In some embodiments, the report comprises one or more diagnostics. In some embodiments, the report comprises lipid profiling.

In some embodiments, the computer processing comprises deconvoluting the spectrum or the metabolome to determine a presence or absence of the plurality of metabolites. In some embodiments, the plurality of metabolites are determined quantitatively.

In another aspect, disclosed herein is a method for providing a risk of a subject having a disease or disorder, comprising: (a) obtaining a biomarker profile of said subject; (b) receiving a request from said subject to process said biomarker profile to identify said risk; (c) processing said request to determine said risk from said biomarker profile of said subject; (d) providing a report to said subject, wherein said report provides said risk of said subject having said disease or disorder, wherein said biomarker profile comprises a metabolomic profile, a genomic profile, a transcriptomic profile, or a proteomic profile.

In another aspect, disclosed herein is a system of processing or analyzing a biological sample of a subject, comprising: one or more computer processors that are individually or collectively programmed to: (i) process a nuclear magnetic resonance (NMR) spectrum of said biological sample of said subject, or a metabolome derived from said NMR spectrum, to determine that said subject has or is at an increased risk of developing a disease, wherein said NMR spectrum or said metabolome corresponds to a plurality of metabolites; and (ii) output a report indicative of said subject having or being at said increased risk of developing said disease.

In another aspect, disclosed herein is a system of processing a biological sample of a subject, comprising: one or more computer processors that are individually or collectively programmed to: (i) process a nuclear magnetic resonance (NMR) spectrum of said biological sample of said subject to identify a metabolome of said biological sample; (ii) process said metabolome to determine that said subject has a disease at a specificity of at least 80%; and (iii) output a report indicative as to whether said subject has or is at said increased risk of developing said disease. In some embodiments, said one or more computer processors are within a cloud computing system.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a workflow of a method for processing a biological sample of a subject to determine whether the subject has or is at an increased risk of developing a disease or disorder.

FIG. 2 depicts use of a nuclear magnetic resonance (NMR) instrument (or spectroscopy) to generate an NMR spectrum or a metabolome derived from the NMR spectrum. The NMR spectrum or the metabolome derived from the NMR spectrum may be processed by the methods described herein to further generate a quantitative, digital representation of the NMR spectrum or of the metabolome derived from the NMR spectrum. The quantitative, digital representation may be used to determine whether a subject has or is at an increased risk of developing a disease or disorder.

FIG. 3 depicts a workflow of methods described herein using a quantitative, digital representation of a metabolome to determine whether a subject has or is at an increased risk of developing a disease or disorder. The subject may be provided a report or personal health assessment indicative of the subject having or being at an increased risk of developing a disease or disorder.

FIG. 4 depicts use of a database having healthy subjects and having subjects with a disease or disorder to generate a health book by the methods described herein. The health book may be used, in part, to predict whether a subject has or is at an increased risk of developing a disease or disorder by the methods described herein.

FIG. 5 depicts use of a health book to predict whether a subject has or is at an increased risk of developing a disease or disorder by the methods described herein.

FIG. 6 depicts use of machine learning methods to determine whether a subject has or is at an increased risk of developing a disease or disorder by the methods described herein.

FIG. 7 depicts a non-limiting example of a computing device configured to perform methods described herein.

FIG. 8 depicts a non-limiting example of a web/mobile application provision system configured to perform methods described herein.

FIG. 9 depicts a non-limiting example of a cloud-based web/mobile application provision system configured to perform methods described herein.

FIGS. 10-19 are flow charts of methods of the present disclosure, according to some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include plural references unless the context clearly dictates otherwise. Any reference to “or” herein is intended to encompass “and/or” unless otherwise stated.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. The term “metabolome,” as used herein, generally refers to a plurality of metabolites. In some cases, the plurality of metabolites can be from a same sample (e.g., from a same subject).

The term “disease,” as used herein, generally refers to an abnormal condition, or a disorder of a biological function or a biological structure such as an organ, that affects part or all of a subject. A disease may be caused by factors originally from an external source, such as infectious disease, or it may be caused by internal dysfunctions, such as autoimmune diseases. A disease can refer to any condition that causes pain, dysfunction, distress, social problems, and/or death to the subject afflicted. A disease may be an acute condition or a chronic condition. A disease may refer to an infectious disease, which may result from the presence of pathogenic microbial agents, including viruses, bacteria, fungi, protozoa, multicellular organisms, and aberrant proteins as prions. A disease may refer to a non-infectious disease, including but not limited to cancer and genetic diseases. In some cases, a disease can be cured. In some cases, a disease cannot be cured. In some cases, the disease can be cancer.

The term “subject,” as used herein, generally refers to an animal, such as a mammal. A subject may be a human or non-human mammal. A subject may be afflicted with a disease or suspected of being afflicted with or having a disease. The subject may not be suspected of being afflicted with or having the disease. The subject may be symptomatic. Alternatively, the subject may be asymptomatic. In some cases, the subject may be treated to alleviate the symptoms of the disease or cure the subject of the disease. A subject may be a patient undergoing treatment by a healthcare provider, such as a doctor.

Many diseases can be linked to metabolism and metabolic disorders thereof. Metabolism can mean, for example, chemical reactions in a human subject's cells that convert food into energy. Metabolites generally mean, for example, chemical products of metabolism that are used for the subject's bodily functions of moving, thinking, or growing. Metabolome generally means, for example, a complete network of metabolites found within a biological sample of the subject.

A metabolome may include, for example, at least 1, 2, 3, 4, 5, 6, 7, 8, 9, 10, 20, 30, 40, 50, 100, 200, 300, 400, 500, 1000, 2000, 3000, or more metabolites. The metabolome may include up to about 100, 200, 300, 400, 500, 1000, 2000, 3000 or more metabolites. The metabolites may be different metabolites. For example, the metabolome includes up to 1000, 2000, or 3000 different metabolites. At least some of these metabolites may be measured, quantified, and digitized to generate a digital metabolic profile of a subject.

A metabolome may be a dynamic network that may mirror an actual, real-time metabolic state of a subject. Further, a metabolome may be a dynamic network that may change with a subject's age and gender. Also, a metabolome may be a dynamic network that may change due to external or internal stimuli such as lifestyle changes. A dynamic network may stand in contrast to a static network. A static network may include a network associated with genomics, transcriptomics, or proteomics. Together, analysis and use of both dynamic and static networks may provide deeper insights into human health for deciphering illness and improving treatment. For example, deeper insights may include deeper insights about diseases or disorders, treatments, or wellness. Deeper insights about diseases or disorders may include, for example, further insights into diagnostics, monitoring flare-ups, or staging of diseases or disorders. Deeper insights about treatment may include, for example, further insights into treatment response, side effects of treatment, or therapeutic dosages for treatment. Deeper insights about wellness may include, for example, health status, pre-screening, or metabolic profiles.

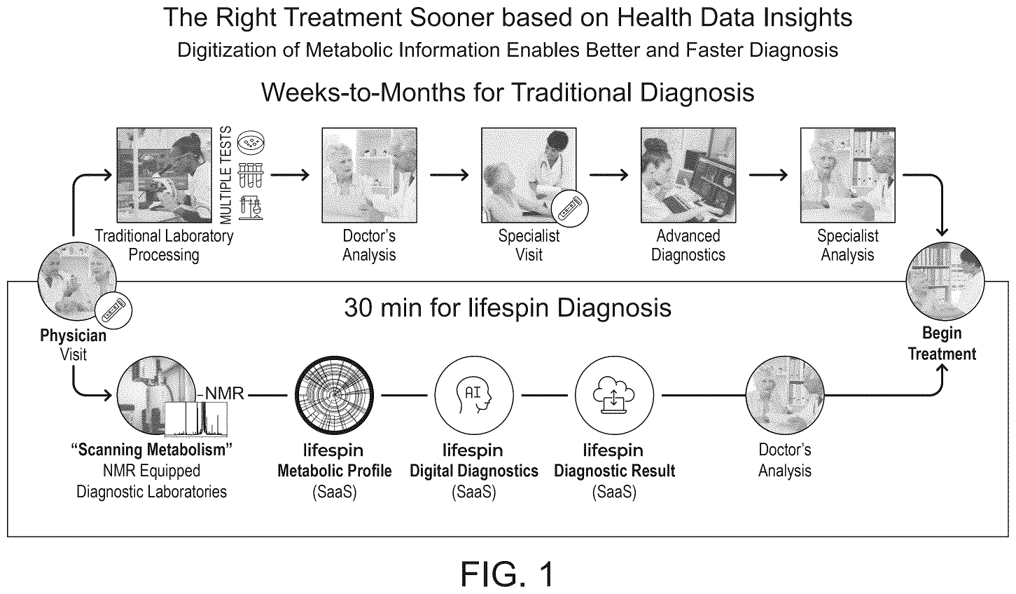

Digital metabolic profiles associated with metabolism and metabolic disorders thereof may exhibit highly distinct deviations based on quantitative measurements compared to healthy metabolic profiles. However, some diseases that can be linked to metabolism and metabolic disorders thereof may take weeks or months to assess before treatment can begin. Compared to the methods described herein, some methods may require many time consuming and costly processes before assessment and treatment for a subject can begin. For example, as shown in FIG. 1, some methods may require an initial consult with a physician. The physician may order one or more tests requiring extensive laboratory processing and analysis. The analysis may then be provided to the physician for further assessment. After further assessment, the physician may require a follow up visit with a specialist. The specialist may then order one or more tests which in turn may be processed and analyzed with advanced diagnostics. The results of these advanced diagnostics may then require further assessment by the specialist. The specialist may determine the disease so the subject having or being at increased risk of developing the disease can begin treatment. Compared to the methods described herein, these many processes are time consuming, costly, and unnecessarily delay treatment.

Diseases that may be linked to metabolism and metabolic disorders thereof may include neurological diseases or disorders. Disease that may be linked to metabolism and metabolic disorders thereof also may include cancers or inflammatory diseases or disorders. For example, neurological diseases or disorders may be the largest cause of disability affecting at least 16% of the world's population. Neurological diseases or disorders may include, for example, neurodegenerative diseases, multiple sclerosis, Parkinson's disease, atypical Parkinson's disease, Alzheimer's disease, or dementia. Multiple sclerosis, which attacks the brain or spinal cord, may affect about 2.8 million people worldwide or about 1 in every 3,000 people.

Such diseases or disorders are difficult to assess due to, for example, unspecific symptoms or slow progression of the disease or disorder. Assessment of neurological diseases or disorders are complex because, for example, symptoms are often unspecific. Assessment of neurological diseases or disorders are slow because, for example, symptoms may take weeks to years for full assessment. For example, steady progression of symptoms may take 10 to 20 years from onset of a neurological disease or disorder. There is a time sensitive window shortly after onset of the neurological disease or disorder in which treatment may be most effective. Unfortunately, due to unspecific symptoms, assessing neurological diseases or disorders during this time sensitive window may not be possible due to a lack of assessment methods for detecting underlying causes of unspecific symptoms. Assessment of neurological diseases or disorders are costly because, for example, assessment may cost up to $50,000 per year or more.

The present disclosure can address at least these issues, for example, by providing methods for determining whether a subject has or is at an increased risk of developing a disease or disorder based at least on using a nuclear magnetic resonance (NMR) instrument to generate an NMR spectrum or a metabolome derived from the NMR spectrum. Methods described herein may process a biological sample of the subject to determine whether the subject has or is at an increased risk of developing a disease or disorder. Methods described herein may identify that the subject has or is suspected of having a disease or disorder. Methods described herein may monitor the subject who has or is suspected of having a disease or disorder. Methods described herein may be performed using machine learning to determine that the subject has or is at an increased risk of developing a disease. Methods described herein may be performed using a cloud computing system to determine that the subject has or is at an increased risk of developing a disease or disorder. Methods described herein may receive a report or provide a report to the subject indicative of the subject having or being at increased risk of developing a disease or disorder with a high level of confidence.

Although methods described herein use a NMR spectrum or a metabolome derived from the NMR spectrum, the methods are not limited to metabolomics. For example, other types of omics may be derived from methods described herein using, in part, a NMR spectrum. Other types of omics that may be derived from a NMR spectrum may include genomics, transcriptomics, or proteomics. In this sense, the methods described herein may provide an integrated or multi-omics approach to determining whether a subject has or is at an increased risk of developing a disease or disorder.

Recognized herein is a need for methods that process a biological sample to determine whether a subject has or is at an increased risk of developing a disease. Some methods described herein May 1) use noninvasive procedures to collect and process a human sample, 2) require inconsequential amounts of a human sample, 3) provide real-time or near real-time analysis of a human sample to assess disease, 4) cost less to assess disease compared to other methods, or 5) provide digital, quantitative measurement of parameters indicative of disease, or 6) predict disease with a high level of confidence. Doing so may provide unique and immediate clinical insights into disease.

### Methods for Predicting a Disease or Disorder

In an aspect, disclosed herein is a method of processing a biological sample of a subject, comprising: (a) using a nuclear magnetic resonance (NMR) instrument to generate an NMR spectrum of the biological sample of the subject; (b) computer processing the NMR spectrum or a metabolome derived from the NMR spectrum to determine that the subject has or is at an increased risk of developing a disease, wherein the NMR spectrum or the metabolome corresponds to a plurality of metabolites; and (c) electronically outputting a report indicative of the subject having or being at the increased risk of developing the disease.

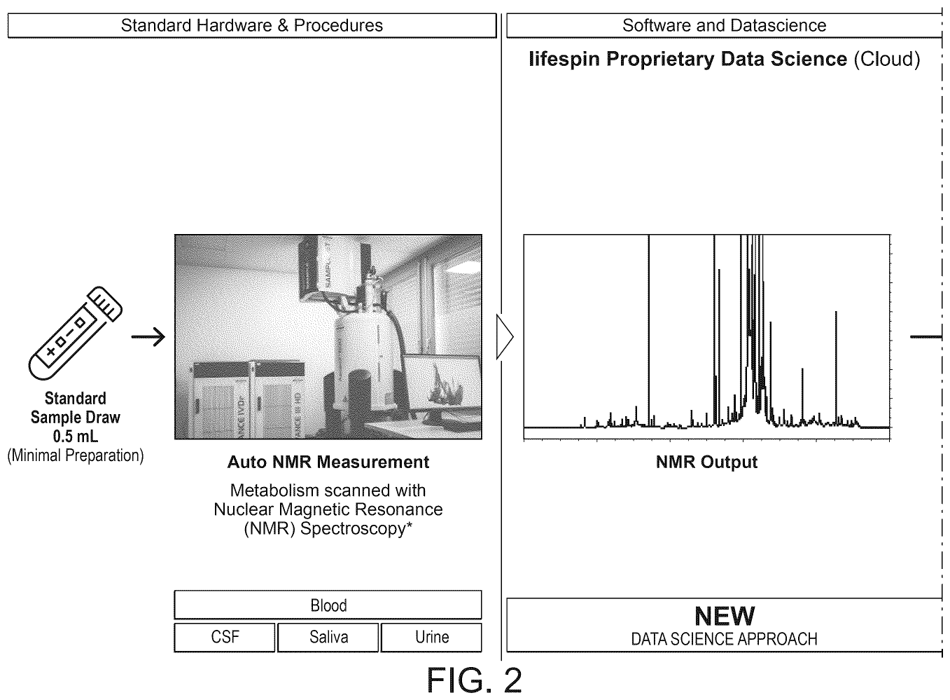

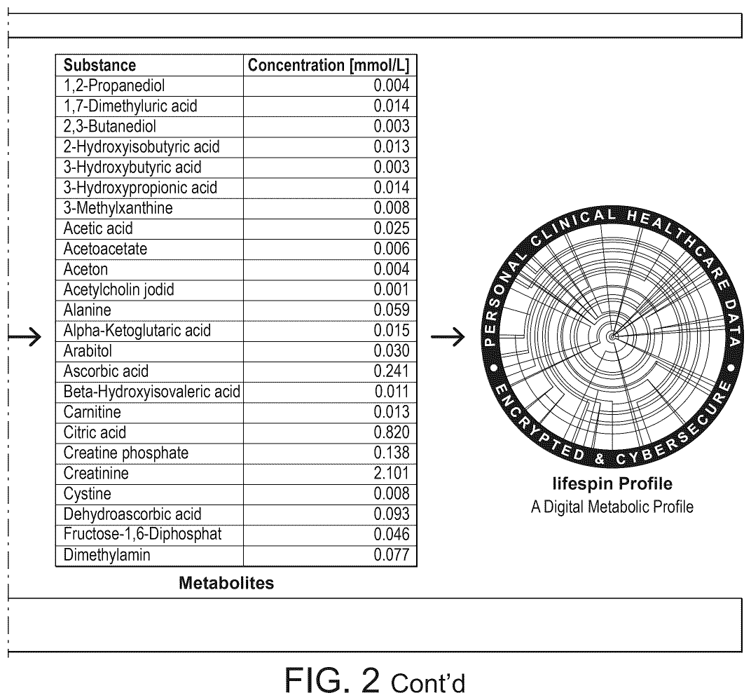

In an example, as shown in FIG. 2, methods disclosed herein may determine whether a subject has or is at an increased risk of developing a disease or disorder. In some embodiments, a time from (a) to (c) is less than about 60 minutes. In some embodiments, a time from (a) to (c) is less than about 30 minutes. In some embodiments, (b) or (c) is performed using a cloud computing system. In some embodiments, (b) and (c) are performed using a cloud computing system. In some embodiments, (b) is performed using a trained machine learning algorithm. In some embodiments, (b) and (c) are performed using one or more trained machine learning algorithms.

Further, as shown, in FIG. 2, a sample draw may be used to collect a sample from the subject. The sample may include, for example, a biological sample of the subject. In some embodiments, the biological sample has not been purified. In some embodiments, the biological sample comprises bile acids, blood (e.g., arterial, capillary, umbilical cord, venous, whole), bronchoalveolar lavage fluid, cell extracts, cerebrospinal fluid, exhaled breath condensates (EBC), plasma (e.g., citrate, EDTA, hirudin, lithium heparin, sodium fluoride), saliva, serum, sperm, stool, sweat, tear fluid, tracheal wash (TW), or urine. In some embodiments, the biological sample is not destroyed. In some embodiments, the biological sample is derived from a human. In some embodiments, the biological sample is derived from an animal.

Further, as shown in FIG. 2, the biological sample may be measured using a NMR instrument to determine, for example, a presence or absence of biomarkers. The biological sample may be measured using a NMR instrument to determine, for example, a quantity of biomarkers. The quantity of biomarkers may be expressed using, for example, a concentration of the biomarkers. The NMR instrument may generate, for example, a NMR spectrum of biomarkers from the presence, absence, or measured quantities of biomarkers. The biomarkers may include, for example, a metabolome having a plurality of metabolites indicative of a subject having or being at an increased risk of developing a disease or disorder. In some embodiments, the plurality of metabolites comprises 3 or more metabolites. In some embodiments, the plurality of metabolites comprises 4 or more metabolites. In some embodiments, the plurality of metabolites comprises 5 or more metabolites. In some embodiments, the plurality of metabolites comprises 10 or more metabolites. In some embodiments, the plurality of metabolites comprises 50 or more metabolites. In some embodiments, the computer processing comprises deconvoluting the spectrum or the metabolome to determine a presence or absence of the plurality of metabolites.

The biomarkers may include, for example, a plurality of ratios of metabolites that are distinctly different than ratios of metabolites for healthy subjects. Alternatively or additionally, other operations may include without limitation, for example, summing, subtracting, or multiplying concentration levels of metabolites. The metabolites may be associated with, for example, a network including other metabolites. The metabolites may be associated with, for example, a network including other biomarkers. The network of other biomarkers may be associated with for example, genomic, transcriptomic, or proteomic networks. The network of metabolites may be analyzed using, for example, artificial intelligence (AI) methods to predict whether a subject has or is at an increased risk of developing a disease or disorder. The AI methods may include, for example, machine learning methods. The predictions of the machine learning methods may be provided to the subject as a report. The network of other biomarkers may be analyzed using, for example, artificial intelligence (AI) methods to predict whether a subject has or is at an increased risk of developing a disease or disorder. The AI methods may include, for example, machine learning methods. The predictions of the machine learning methods may be provided to the subject as a report. Together, the network of metabolites and the network of other biomarkers may be analyzed using, for example, artificial intelligence (AI) methods to predict whether a subject has or is at an increased risk of developing a disease or disorder. The AI methods may include, for example, machine learning methods. The predictions of the machine learning methods may be provided to the subject as report.

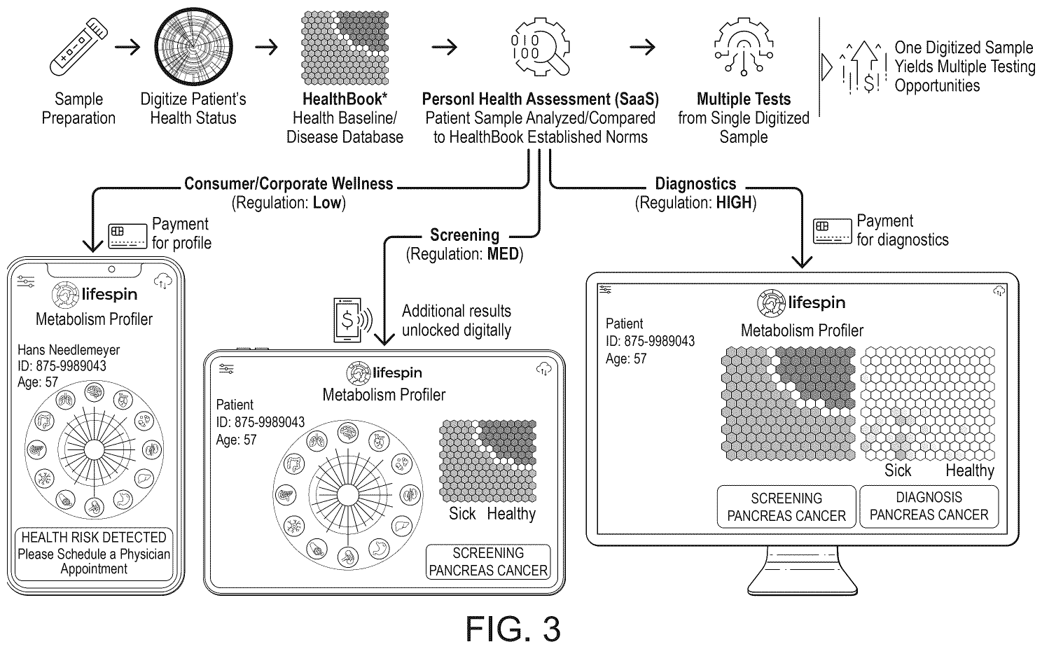

In an example, as shown in FIG. 3, the predictions may be provided as a report, for example, a personal health assessment report. In some embodiments, the report has an accuracy, selectivity, or specificity of at least about 80%, 85%, 90%, or greater. In some embodiments, the report comprises one or more diagnostics. The personal health assessment report may include, for example, reports having different levels of detail. For example, the personal health assessment report may provide a low level of detail sufficient for indicating wellness of the subject. The report having a low level of detail may recommend that the subject schedule an appointment with a physician for further assessment to determine, for example, whether the subject has cancer, a metabolic disease or disorder, an inflammatory disease or disorder, or a neurological disease or disorder. The personal health assessment report may provide a medium level of detail sufficient for screening purposes. The report having a medium level of detail may recommend that the subject be screened for a disease or disorder, for example, a neurological disease or disorder. The report may provide a high level of detail sufficient to indicate that the subject has or is at an increased risk of developing a disease. The report having a high level of detail may diagnose that the subject has a specific disease or disorder, for example, Parkinson's disease. In some embodiments, the report comprises lipid profiling. Lipid profiling may include lipids such as fatty acids, glycerolipids, phospholipids, sterols, ceramides, sphingolipids, acyl-carnitines, or lipoproteins.

In an example, as shown in FIGS. 2 and 3, methods disclosed herein may provide quantitative, digital scanning and identification of an NMR spectrum or a metabolome derived from the NMR spectrum to determine that a subject has or is at an increased risk of developing a disease or disorder. The biological sample may be, for example, a standard sample draw of about 0.5 mL of blood from the subject. Alternatively or additionally, the biological sample may include, for example, urine, saliva, cerebrospinal fluid, serum, EDTA-plasma, citrate-plasma, hirudin-plasma, bile acids, cell extracts, or bronchoalveolar lavage fluid. The biological sample may require minimal preparation for use in an NMR instrument. For example, the biological sample may be transferred to an NMR tube and mixed with deuterated locking solvent. An NMR instrument may operate at frequencies of up to about 1.2 GHz. An NMR instrument may operate at magnetic field strengths of up to about 28.2 Tesla (T).

Further, as shown in FIG. 3, the biological sample may be scanned by the NMR instrument using NMR spectroscopy techniques to determine the NMR spectrum or a metabolome derived from the NMR spectrum. The NMR spectrum may include, for example, presence, absence, or quantities of metabolites. The NMR spectrum may include, for example, presence, absence, or quantities of substances other than metabolites. The substances other than metabolites may include, for example, substances associated with genomics, transcriptomics, or proteomics. Quantities of metabolites may include, for example, concentrations of the metabolites. Quantities of substances other than metabolites may include, for example, concentrations of the substances other than metabolites. The metabolome may include, for example, the concentrations of the metabolites. In some embodiments, the metabolome is a full metabolome. In some embodiments, the metabolome is a full metabolome to a detection limit of the NMR instrument. A detection limit may comprise, for example, concentrations of metabolites of less than about 100 micromolar (μM), 10 μM, 1 μM or less.

Further, as shown in FIG. 3, the concentrations of metabolites may be digitized to create a digital metabolic profile of the subject. The digital metabolic profile may further include ratios of concentrations of metabolites. Alternatively or additionally, other operations may include without limitation, for example, summing, subtracting, or multiplying concentration levels of metabolites. The digital metabolic profile may then be assessed to determine whether the subject has or is at an increased risk of developing a disease or disorder. For example, the digital metabolic profile may be assessed by comparing concentrations of metabolites for the subject to concentrations of metabolites for healthy subjects. Alternatively or additionally, the digital metabolic profile may be assessed by comparing concentrations of metabolites for the subject to concentrations of metabolites for subjects known to have a disease or disorder. For example, the digital metabolic profile may be assessed by comparing ratios of concentrations of metabolites for the subject to ratios of concentrations of metabolites for healthy subjects. Alternatively or additionally, the digital metabolic profile may be assessed by comparing ratios of concentrations of metabolites for the subject to ratios of concentrations of metabolites for subjects known to have a disease or disorder. Comparing ratios of concentrations of metabolites may, for example, provide quantitative biomarkers for specific diseases or disorders.

Concentrations of metabolites or ratios of concentrations of metabolites thereof may be compared using, for example, artificial intelligence (AI) methods to predict whether a subject has or is at an increased risk of developing a disease or disorder. The AI methods may include, for example, machine learning methods. The machine learning methods may include, for example, machine learning algorithms. In some embodiments, the trained machine learning algorithm is trained using at least 1000 biological samples.

Machine learning algorithms may be implemented in, for example, programming languages. Programming languages may include, for example, R (e.g., R Core Team©), Python®, TensorFlow®, Java®, JavaScript®, C/C++, C#, Julia®, Scala®, Octave®, Matlab®, or SAS®. The machine learning algorithms may be trained using, for example, different types of algorithms. Different types of algorithms may include, for example, linear regression, logistic or nonlinear regression, decision trees, random forests, support vector machines, K-means clustering, K-nearest neighbors, principal components analysis, artificial neural networks, or self-organizing maps. In some embodiments, the trained machine learning algorithm comprises use of one or more artificial neural networks, random forests, support vector machines, linear regressions, nonlinear regressions, or clusterings. In some embodiments, the trained machine learning algorithm comprises use of the one or more artificial neural networks. In some embodiments, the one or more artificial neural networks comprises use of one or more self-organizing maps.

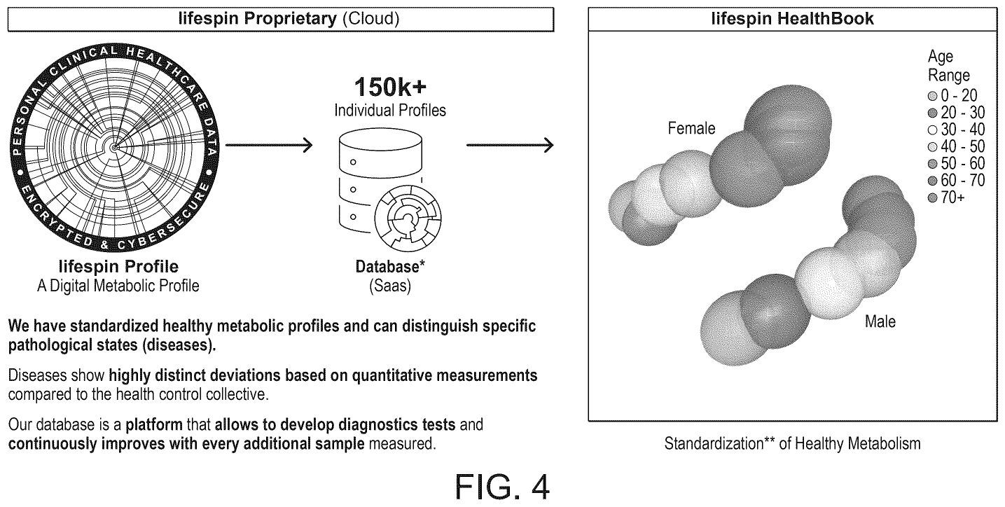

In an example, as shown in FIG. 4, the concentration levels of healthy subjects or of subjects having a disease or disorder may include metabolomes related to at least up to about 400, 14,000, 75,000, 150,000, 250,000, 500,000, 1.5 million, 3 million, 6 million, or 10 million individual subjects or more. For example, the metabolomes may be related to at least about 100,000 individual subjects.

In some embodiments, the metabolome comprises information related to a number of at least about 50 metabolites. In some embodiments, the metabolome comprises information related to a number of at least about 100 metabolites. In some embodiments, the metabolome comprises information related to a number of at least about 500 metabolites. In some embodiments, the metabolome is not deconvoluted.

Further, as shown in FIG. 4, the metabolomes may be stored in a database, for example, a relational database, a non-relational database, a semi-relational database, or a combination thereof. The database may be implemented, for example, as a software as a service (SaaS), a platform as a service (PaaS), or an infrastructure as a service (IaaS), or a combination thereof. The database may include features associated with subjects and metabolomes. Features may include up to at least about 10, 50, 100, or more features. The features may be used by methods described herein to determine whether a subject has or is at an increased risk of developing a disease or disorder. Features may include, for example, metadata about the individual subjects. The metadata may include, for example, gender, age, weight, ethnicity, health status, clinical metadata, or non-clinical metadata, or any combination thereof. Features may include, for example, concentrations of metabolites or ratios of concentrations of metabolites. Features may include, for example, data associated with other than concentrations of metabolites or ratios of concentrations of metabolites. Features may include, for example, data associated with lipoproteins and subclasses of lipoproteins. The database may be populated using different sources of information. Different sources may include, for example, public and private repositories of data associated with other individual healthy subjects or other individual subjects having a disease or disorder. In some embodiments, the machine learning comprises use of a database comprising at least about 100,000 NMR spectra related to other metabolomes. The spectra may include, for example, 1D spectra, 2D spectra, or both.

Further, as shown in FIG. 4, the metabolomes of the individual subjects may be labeled by, for example, labeling individual subjects as healthy subjects. The metabolomes of the individual subjects may be labeled by, for example, labeling individual subjects as having a disease or disorder. The metabolomes of the individual subjects may be standardized or normalized.

Machine learning methods may be trained using metabolomes related to, for example, at least up to the about 100,000 individual subjects or more. Machine learning methods may be trained using, for example, a supervised method by training the machine learning algorithm with the individual subjects labeled as healthy subjects or individual subjects labeled as having a disease or disorder. Machine learning methods may be trained using, for example, an unsupervised method by training the machine learning algorithm with individual subjects not labeled as healthy subjects or with individual subjects not labeled as subjects having a disease or disorder. Machine learning methods may be trained using, for example, supervised and unsupervised methods. By comparing concentrations of metabolites, the machine learning methods may then generate, for example, a personal health assessment of the subject described elsewhere herein. By comparing ratios of concentrations of metabolites, the machine learning methods may then generate, for example, a personal health assessment of the subject described elsewhere herein. Alternatively or additionally, other operations may include without limitation, for example, summing, subtracting, or multiplying concentration levels of metabolites. The personal health assessment may, for example, predict a disease or disorder, distinguish between diseases or disorders, or otherwise indicate whether the subject has or is at an increased risk of developing a disease or disorder. The personal health assessment may, for example, identify the subject as having a disease or disorder. The personal health assessment may, for example, monitor progression or regression of the subject having a disease or disorder.

The machine learning methods may make predictions with, for example, a performance having area under curve (AUC) of a receiver operating characteristic (ROC) curve. The ROC curve may be expressed as, for example, true positive rate (TPR) versus false positive rate (FPR). The AUC may be, for example, at least about 0.5, 0.6, 0.7, 0.8, 0.9, or greater. The machine learning methods may make predictions with, for example, a sensitivity. The sensitivity may be, for example, at least about 0.5, 0.6, 0.7, 0.8, 0.9, or greater. The machine learning methods may make predictions with, for example, a specificity. The specificity may be, for example, at least about 0.5, 0.6, 0.7, 0.8, 0.9, or greater. The machine learning methods may make predictions with, for example, a negative predictive value (NPV). The NPV may be, for example, at least about 0.5, 0.6, 0.7, 0.8, 0.9, or greater. The machine learning methods may make predictions with, for example, a positive predictive value (PPV). The PPV may be, for example, at least about 0.5, 0.6, 0.7, 0.8, 0.9, or greater. The machine learning methods may make predictions with, for example, an accuracy. The accuracy may be, for example, at least about 0.5, 0.6, 0.7, 0.8, 0.9, or greater.

For example, the machine learning methods may predict a single type of disease or disorder with an AUC. The machine learning methods may make predictions for multiple sclerosis having, for example, an AUC of at least about 0.5, 0.6, 0.7, 0.8, 0.9, or greater. The machine learning methods may make predictions for Parkinson's disease having, for example, an AUC of at least about 0.5, 0.6, 0.7, 0.8, 0.9, or greater.

For example, the machine learning methods may distinguish between two or more types of diseases or disorders with an AUC of an ROC curve. For example, the machine learning methods may distinguish between multiple sclerosis and other types of diseases or disorders. The machine learning methods may distinguish between multiple sclerosis and degenerative diseases with an AUC of at least about 0.5, 0.6, 0.7, 0.8, 0.9, or greater. The machine learning methods may distinguish between multiple sclerosis and neurodegenerative disease of the nervous system with an AUC of at least about 0.5, 0.6, 0.7, 0.8, 0.9, or greater. about 0.5, 0.6, 0.7, 0.8, 0.9, or greater. The machine learning methods may distinguish between multiple sclerosis and primary Parkinson's syndrome with an AUC of at least about 0.5, 0.6, 0.7, 0.8, 0.9, or greater. about 0.5, 0.6, 0.7, 0.8, 0.9, or greater. The machine learning methods may distinguish between multiple sclerosis and dementia with an AUC of at least about 0.5, 0.6, 0.7, 0.8, 0.9, or greater. about 0.5, 0.6, 0.7, 0.8, 0.9, or greater. The machine learning methods may distinguish between multiple sclerosis and Alzheimer's disease with an AUC of at least about 0.5, 0.6, 0.7, 0.8, 0.9, or greater. about 0.5, 0.6, 0.7, 0.8, 0.9, or greater. The machine learning methods may distinguish between relapsing multiple sclerosis and progressive multiple sclerosis.

For example, the machine learning methods may distinguish between Parkinson's disease and other types of diseases or disorders. The machine learning methods may distinguish between Parkinson's disease and degenerative diseases other than Parkinson's disease with an AUC of at least about 0.5, 0.6, 0.7, 0.8, 0.9, or greater. The machine learning methods may distinguish between Parkinson's disease and dementia with an AUC of at least about 0.5, 0.6, 0.7, 0.8, 0.9, or greater. The machine learning methods may distinguish between Parkinson's disease and neurodegenerative diseases with an AUC of at least about 0.5, 0.6, 0.7, 0.8, 0.9, or greater. The machine learning methods may distinguish between Parkinson's disease and cerebral infarctions with an AUC of at least about 0.5, 0.6, 0.7, 0.8, 0.9, or greater. The machine learning methods may distinguish between Parkinson's disease and Alzheimer's disease with an AUC of at least about 0.5, 0.6, 0.7, 0.8, 0.9, or greater.

For example, the machine learning methods may make predictions using gender as a feature among other features. The machine learning methods may predict whether the subject, being a male subject, has or is at an increased risk of developing a disease or disorder (e.g., multiple sclerosis) with an area under curve (AUC) of at least about 0.5, 0.6, 0.7, 0.8, 0.9, or greater. The machine learning methods may predict whether the subject, being a female subject, has or is at an increased risk of developing a disease or disorder (e.g., multiple sclerosis) with an area under curve (AUC) of at least about 0.5, 0.6, 0.7, 0.8, 0.9, or greater. Differences between males and females may result from, for example, different responses to inflammation, disturbed energy metabolism (e.g., oxidative stress, mitochondrial dysfunction, or degeneration), gut-brain axis (e.g., enrichment of intestinal floral product), or disturbed fatty acid metabolism.

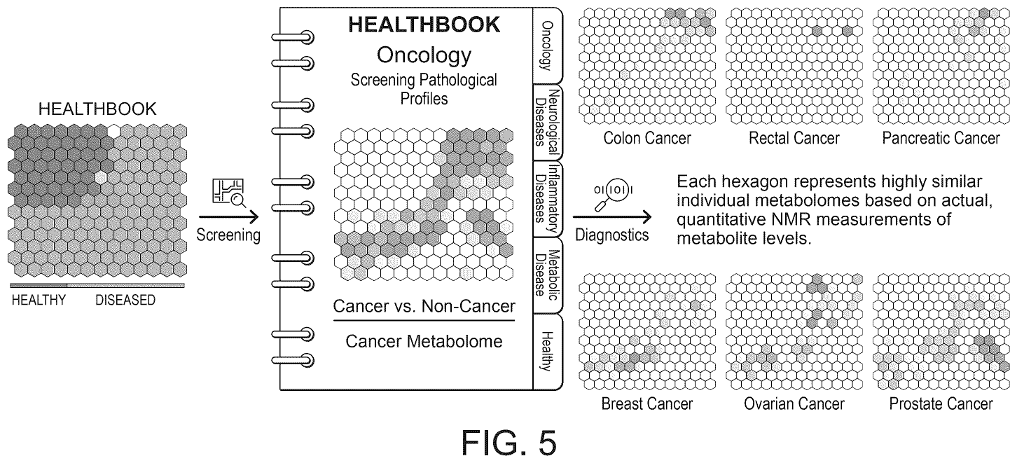

In an example, as shown in FIG. 5, the database having the metabolomes of up to at least about 100,000 individual subjects or more may be associated with a health book. The health book may be generated, in part, from the database by machine learning methods described herein. The health book may include, for example, multidimensional data of metabolite concentrations. The health book may include, for example, multidimensional data of ratios of metabolite concentrations. The health book may include, for example, metabolite concentrations or ratios of metabolite concentrations for a number of metabolites up to at least about 50, 100, 1000, 3000 or more metabolites. Alternatively or additionally, other operations may include without limitation, for example, summing, subtracting, or multiplying concentration levels of metabolites. The health book may include, for example, metadata. The metadata may include, for example, gender, age, weight, ethnicity, health status, clinical metadata, or non-clinical metadata, or any combination thereof.

The health book may include, for example, metabolomes that are related to healthy metabolomes or metabolomes that are related to metabolomes having a disease or disorder. The health book may be associated with diseases or disorders. The diseases or disorders may include, for example, cancers, metabolic diseases or disorders, inflammatory diseases or disorders, or neurological diseases or disorders.

In some embodiments, the disease is a cancer. In some embodiments, the cancer comprises lung cancer, thyroid cancer, colorectal cancer, breast cancer, prostate cancer, rectal cancer, chronic lymphatic leukemia, pancreatic cancer, or ovarian cancer. In some embodiments, the disease is a metabolic disease. In some embodiments, the metabolic disease comprises kidney disease or liver disease. In some embodiments, the disease is an inflammatory disease. In some embodiments, the inflammatory disease comprises rheumatoid arthritis or ulcerative colitis. In some embodiments, the disease is a neurological disease. In some embodiments, the neurological disease comprises multiple sclerosis, Parkinson's disease, or Alzheimer's disease.

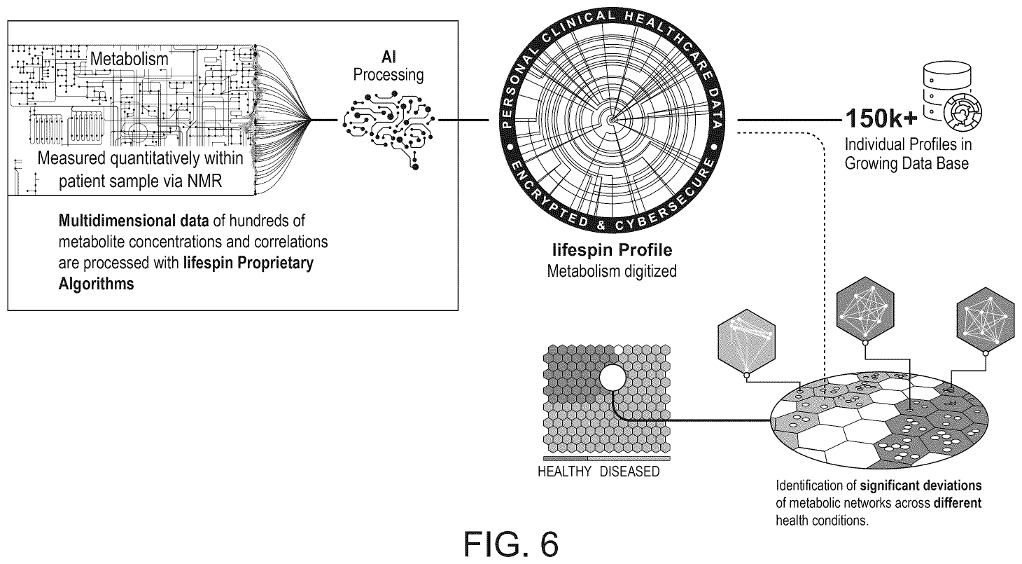

In an example, as shown in FIG. 6, the machine learning methods may be trained using data in the database. The machine learning methods may generate, in part, a health book. The machine learning methods may use a health book to compare concentrations of metabolites to determine whether a subject has or is at increased risk of developing a disease or disorder. The machine learning methods may use a health book to compare ratios of concentrations of metabolites to determine whether a subject has or is at increased risk of developing a disease or disorder.

Further, as shown in FIG. 6, the health book may be associated with methods using artificial intelligence (AI) to generate the health book from the database. Methods using AI may be associated with methods using, for example, machine learning (ML). Methods using ML may be associated with methods using, for example, artificial neural networks (ANN). Methods using ANN may be associated with methods using, for example, self-organizing maps (SOM). The ML may be associated with, for example, low-dimensional, discretized representations of inputs. The inputs may be associated with, for example, metabolomes of individual subjects. The metabolomes may be associated with, for example, concentrations of metabolites or ratios of concentrations of metabolites of individual subjects. Alternatively or additionally, other operations may include without limitation, for example, summing, subtracting, or multiplying concentration levels of metabolites. The metabolomes may be associated with healthy subjects. The metabolomes may be associated with subjects having a disease or disorder. The inputs may be associated with, for example, metadata of individual subjects. The metadata may be associated with, for example, gender, age, weight, ethnicity, health status, clinical metadata, or non-clinical metadata, or any combination thereof.

Further, as shown in FIG. 6, the inputs may be associated with outputs. The outputs may be, for example, a clustered representation indicating concentrations of metabolites associated with subjects having a disease or disorder. The outputs may be, for example, a clustered representation indicating ratios of concentrations of metabolites associated with subjects having disease or disorder. The outputs may be, for example, a clustered representation indicating concentrations of metabolites associated with healthy subjects. The outputs may be, for example, a clustered representation indicating ratios of concentrations of metabolites associated with healthy subjects. The outputs may include, for example, metadata. Metadata may include, for example, gender, age, weight, ethnicity, health status, clinical metadata, or non-clinical metadata, or any combination thereof. The outputs may be used to generate, in part, the health book.

The clustered representations may be clustered by, for example, recognizing significant deviations of metabolomes across different diseases or disorders. In some embodiments, the machine learning comprises determining a deviation of the NMR spectrum or the metabolome from a healthy set of information stored in the database. Deviations may include, for example, deviations in concentrations of metabolites between healthy subjects and subjects having a disease or disorder. Deviations may include, for example, deviations in ratios of concentrations of metabolites between healthy subjects and subjects having a disease or disorder. Alternatively or additionally, other operations may include without limitation, for example, summing, subtracting, or multiplying concentration levels of metabolites. Deviations may include, for example, deviations across gender, age, weight, or ethnicity or a combination thereof between healthy subjects and subjects having a disease or disorder. Clustering may include similar metabolic profiles associated with healthy subjects. Clustering may include similar metabolic profiles associated with subjects having a disease or disorder. The subject, having a metabolome that is measured and quantitatively digitized (as described elsewhere herein), may be determined by methods described herein to have or be at increased risk of developing a disease or disorder by comparing the subject's metabolome to the clustered representations of the health book.

Methods for Predicting a Disease or Disorder with a Prescribed Performance

In another aspect, disclosed herein is a method of processing a biological sample of a subject, comprising: (a) using a nuclear magnetic resonance (NMR) instrument to generate an NMR spectrum of the biological sample of the subject; (b) computer processing the NMR spectrum to identify a metabolome of the biological sample; (c) computer processing the metabolome to determine that the subject has a disease at a specificity of at least 80%; and (d) electronically outputting a report indicative as to whether the subject has or is at the increased risk of developing the disease.

In an example, the methods described herein elsewhere may predict that the subject has a disease or disorder with a prescribed specificity. The prescribed specificity may be, for example, at least 80%. In an example, the methods described elsewhere herein may predict that the subject has a disease or disorder using, for example, an area under curve (AUC) of a receiver operating characteristic (ROC) curve, a true negative rate (TNR), a false positive rate (FPR), a negative predictive value (NPV), a positive predictive value (PPV), or a sensitivity.

In an example, the methods described herein elsewhere may be performed in a prescribed time. In some embodiments, a time from (a) to (d) is less than about 60 minutes. In some embodiments, a time from (a) to (d) is less than about 30 minutes.

In an example, the methods described herein elsewhere may be performed using a cloud computing system. In some embodiments, (b), (c) or (d) is performed using a cloud computing system. In some embodiments, (b), (c) and (d) are performed using a cloud computing system.

In an example, the methods described herein elsewhere may be performed using a trained machine learning algorithm. In an embodiment, (b) or (c) is performed using a trained machine learning algorithm. In an embodiment, (b), (c) or (d) is performed using one or more trained machine learning algorithms.

Methods for Predicting a Disease or Disorder Using a Cloud Computing System from Generated NMR Spectrum

In another aspect, disclosed herein is a method of processing a biological sample of a subject, of the biological sample of the subject; (b) transmitting the NMR spectrum or a metabolome derived from the NMR spectrum to a cloud computing system, which cloud computing system processes the NMR spectrum or the metabolome to determine that the subject has or is at an increased risk of having a disease; and (c) receiving an electronic report indicative of the subject having or being at the increased risk of developing the disease.

In an example, the methods described herein elsewhere may predict that the subject has a disease or disorder using a cloud computing system. The cloud computing system may process the NMR spectrum or the metabolome using a trained machine learning algorithm described elsewhere herein.

In an example, a NMR instrument in a first location may be used to generate an NMR spectrum. The NMR spectrum or a metabolome derived from the NMR spectrum may be transmitted to a second location over a network. The second location may be, for example, a location associated with the cloud computing system. The cloud computing system, or a trained machine learning algorithm associated with the cloud computing system, may process the NMR spectrum or the metabolome to determine that the subject has or is at an increased risk of having a disease or disorder. The cloud computing system may then transmit an electronic report back to the first location or another location.

In an example, the methods described herein elsewhere may be performed in a prescribed time. In some embodiments, a time from (a) to (c) is less than about 60 minutes. In some embodiments, a time from (a) to (c) is less than about 30 minutes.

Methods for Predicting a Disease or Disorder Using a Cloud Computing System from Received NMR Spectrum

In another aspect, disclosed herein is a method of processing a biological sample of a subject, comprising: (a) receiving a nuclear magnetic resonance (NMR) spectrum or a metabolome derived from the NMR spectrum through a cloud computing system, wherein the NMR spectrum is generated from the biological sample of the subject, and wherein the cloud computing system processes the NMR spectrum or the metabolome to determine that the subject has or is at an increased risk of having a disease; and (b) transmitting an electronic report indicative of the subject having or being at the increased risk of developing the disease.

In an example, the methods described herein elsewhere may predict that the subject has a disease or disorder using a cloud computing system. The cloud computing system may process the NMR spectrum or the metabolome using a trained machine learning algorithm described elsewhere herein.

In an example, a first location may request an assessment from a cloud computing system that processes a NMR spectrum or a metabolome to determine that the subject has or is at an increased risk of having a disease or disorder. The cloud computing system, or a trained machine learning algorithm associated with the cloud computing system, may process the NMR spectrum or the metabolome to determine that the subject has or is at an increased risk of having a disease or disorder. The first location may not have a NMR instrument to generate an NMR spectrum or a metabolome derived from the NMR spectrum. The first location may not have an ability to process the NMR spectrum or the metabolome using a trained machine learning algorithm. The cloud computing system or the cloud computing system having a trained machine learning algorithm may transmit an electronic report to the first location or another location. The electronic report may be indicative of the subject having or being at increased risk of developing the disease or disorder.

In an example, the methods described herein elsewhere may be performed in a prescribed time. In some embodiments, a time from (a) to (b) is less than about 60 minutes. In some embodiments, a time from (a) to (b) is less than about 30 minutes.

### Methods for Predicting a Disease or Disorder Using Machine Learning

In another aspect, disclosed herein is a method, comprising: (a) receiving a plurality of nuclear magnetic resonance (NMR) spectra, wherein the plurality of NMR spectra are of a plurality of training samples from a plurality of subjects having been identified as having at least one disease; and (b) using the plurality of NMR spectra or a plurality of metabolomes derived from the plurality of NMR spectra to train a machine learning algorithm to identify at least one feature of the plurality of NMR spectra or the plurality of metabolomes, to yield a trained machine learning algorithm that is configured to (i) take as input an NMR spectrum of a biological sample of a subject or a metabolome derived from the NMR spectrum and (ii) process the NMR spectrum or the metabolome to determine that the subject has or is at an increased risk of having a disease of the at least one disease.

In an example, the methods described herein elsewhere may predict that the subject has a disease or disorder using a trained machine learning algorithm. The trained machine learning algorithm may be implemented using a cloud computing system described elsewhere herein. In an embodiment, (a) or (b) is performed using a cloud computing system. In an embodiment, (a) and (b) are performed using a cloud computing system.

In an example, the methods described herein elsewhere may be performed in a prescribed time. In some embodiments, a time from (b) (i) to (b) (ii) is less than about 60 minutes. In some embodiments, a time from (b) (i) to (b) (ii) is less than about 30 minutes.

### Methods for Monitoring Health or Physiological Condition

In another aspect, disclosed herein is a method for monitoring a health or physiological condition of a subject, comprising: (a) obtaining a first NMR or metabolome spectrum of the subject at a first time point; (b) obtaining a second NMR or metabolome spectrum of the subject at a second time point; and (c) computer processing the first NMR or metabolome spectrum and second first NMR or metabolome spectrum, thereby monitoring the health or physiological condition of the subject.

In an example, the methods described herein elsewhere may monitor a health or physiological condition of the subject using a computer processor. The computer processor may be associated with a cloud computing system. The computer processor may process the NMR spectrum or the metabolome using a trained machine learning algorithm described elsewhere herein.

In an example, the one or more different NMR spectrums or metabolomes may indicate progression or regression of the health or physiological condition of the subject. In some embodiments, the subject has or is suspected of having a disease, and wherein monitoring the health or physiological condition of the subject comprises monitoring a progression or regression of the disease. In an example, the progression may show an improvement in the health or physiological condition of the subject. The regression may show a worsening in the health or physiological condition of the subject. There may be no progression or regression in the health or physiological condition of the subject.

In an example, the computer processor may process the NMR spectrum or the metabolome of the subject over a period of time to monitor the health or physiological condition of the subject. The period of time may be associated with one or more different NMR spectrums or metabolomes of the same subject. In an example, the period of time may be a prescribed period of time for generating one or more NMR spectrums or metabolomes of the same subject. In some embodiments, the second time point occurs about 1 week, 1 month, 1 year, or more after the first time point.

Methods for Identifying Health or Physiological Condition from Received NMR Spectrum

In another aspect, disclosed herein is a method for identifying a health or physiological condition of a subject, comprising: (a) receiving, from a digital computer over a computer network, an NMR or metabolome spectrum of the subject; (b) computer processing the NMR or metabolome spectrum to identify the health or physiological condition of the subject; and (c) transmitting, to the digital computer over the computer network, an electronic report indicative of the health or physiological condition of the subject identified in (b).

In an example, the methods described herein elsewhere may identify that the subject has a disease or disorder using a computer processor. The computer processor may be associated with a cloud computing system. The computer processor may process the NMR spectrum or the metabolome using a trained machine learning algorithm described elsewhere herein.

In an example, the NMR spectrum or metabolome may be used, in part, to identify a health or physiological condition of the subject. In some embodiments, the subject has or is suspected of having a disease, and wherein identifying the health or physiological condition of the subject comprises identifying the disease.

In an example, an NMR instrument in a first location may be used to generate an NMR spectrum or a metabolome derived from the NMR spectrum. The NMR spectrum or the metabolome derived from the NMR spectrum may be transmitted to a second location over a network. The second location may be, for example, a location associated with the cloud computing system. The cloud computing system, or a trained machine learning algorithm associated with the cloud computing system, may process the NMR spectrum or the metabolome to identify that the subject has a disease or disorder. The cloud computing system may then transmit an electronic report back to the first location. The report may identify that the subject has the disease or disorder.

Methods for Identifying Health or Physiological Condition from Generated NMR Spectrum

In another aspect, disclosed herein is a method for identifying a health or physiological condition of a subject, comprising: (a) processing a biological sample of the subject to generate an NMR or metabolome spectrum of the subject; (b) transmitting, over a computer network, the NMR or metabolome spectrum of the subject to a computer system that (i) receives the NMR or metabolome spectrum of the subject and (ii) processes the NMR or metabolome spectrum of the subject to identify the health or physiological condition of the subject; and (c) receiving, over the computer network, an electronic report indicative of the health or physiological condition of the subject identified in (b).

In an example, the methods described herein elsewhere may identify that the subject has a disease or disorder using a computer processor. The computer processor may be associated with a cloud computing system. The computer processor may process the NMR spectrum or the metabolome using a trained machine learning algorithm described elsewhere herein.

In an example, the NMR spectrum or metabolome may be used, in part, to identify a health or physiological condition of the subject. In some embodiments, the subject has or is suspected of having a disease, and wherein identifying the health or physiological condition of the subject comprises identifying the disease.

In an example, a first location may request an assessment from a cloud computing system that processes a NMR spectrum or a metabolome to identify that the subject has a disease. The first location may not have an ability to process the NMR spectrum or the metabolome using a trained machine learning algorithm to identify the subject as having the disease. The cloud computing system, or a trained machine learning algorithm associated with the cloud computing system, may process the NMR spectrum or the metabolome to identify that the subject has the disease. The first location may receive a report from the cloud computing system or the cloud computing system having a trained machine learning algorithm. The report may identify that the subject has the disease or disorder.

Methods for Predicting a Subject as not Having or not being at Risk of Developing a Disease

In another aspect, disclosed herein is a method of processing a biological sample of a subject, comprising: (a) using a nuclear magnetic resonance (NMR) instrument to generate an NMR spectrum of the biological sample of the subject; (b) computer processing the NMR spectrum or a metabolome derived from the NMR spectrum to determine that the subject does not have or is not at an increased risk of developing a disease, wherein the NMR spectrum or the metabolome corresponds to a plurality of metabolites; and (c) electronically outputting a report indicative of the subject not having or not being at the increased risk of developing the disease.

In an example, the methods described herein elsewhere may predict that the subject does not have a disease or disorder. In an example, the methods described herein elsewhere may be performed in a prescribed time. In some embodiments, a time from (a) to (c) is less than about 60 minutes. In some embodiments, a time from (a) to (c) is less than about 30 minutes.

In an example, the methods described herein elsewhere may be performed using a cloud computing system. In some embodiments, (b) or (c) is performed using a cloud computing system. In some embodiments, (b) and (c) are performed using a cloud computing system.

In an example, the methods described herein elsewhere may be performed using a trained machine learning algorithm. In an embodiment, (b) is performed using a trained machine learning algorithm. In an embodiment, (b) and (c) are performed using one or more trained machine learning algorithms.

### Method for Providing a Risk of a Subject Having a Disease or Disorder

In another aspect, disclosed herein is a method for providing a risk of a subject having a disease or disorder, comprising: (a) obtaining a biomarker profile of said subject; (b) receiving a request from said subject to process said biomarker profile to identify said risk; (c) processing said request to determine said risk from said biomarker profile of said subject; (d) providing a report to said subject, wherein said report provides said risk of said subject having said disease or disorder, wherein said biomarker profile comprises a metabolomic profile, a genomic profile, a transcriptomic profile, or a proteomic profile.

In an example, methods disclosed herein may provide a risk of a subject having a disease or disorder from a biomarker profile. The biomarker profile of the subject may include, for example, a metabolomic profile, a genomic profile, a transcriptomic profile, or a proteomic profile. Machine learning methods described herein elsewhere may process one or more biomarker profiles to provide a risk of the subject having the disease or disorder. The machine learning methods may provide a report to the subject of the risk of having the disease or disorder.

### System of Processing or Analyzing a Biological Sample of a Subject

In another aspect, disclosed herein is a system of processing or analyzing a biological sample of a subject, comprising: one or more computer processors that are individually or collectively programmed to: (i) process a nuclear magnetic resonance (NMR) spectrum of said biological sample of said subject, or a metabolome derived from said NMR spectrum, to determine that said subject has or is at an increased risk of developing a disease, wherein said NMR spectrum or said metabolome corresponds to a plurality of metabolites; and (ii) output a report indicative of said subject having or being at said increased risk of developing said disease.

In an example, methods described herein may be implemented by a computing system having one or more computer processors that are individually or collectively programmed.

In another aspect, disclosed herein is a system of processing a biological sample of a subject, comprising: one or more computer processors that are individually or collectively programmed to: (i) process a nuclear magnetic resonance (NMR) spectrum of said biological sample of said subject to identify a metabolome of said biological sample; (ii) process said metabolome to determine that said subject has a disease at a specificity of at least 80%; and (iii) output a report indicative as to whether said subject has or is at said increased risk of developing said disease. In some embodiments, said one or more computer processors are within a cloud computing system.

In an example, methods described herein may be implemented by a computing system having one or more computer processors that are individually or collectively programmed to achieve a specificity of at least 80%.

### Computing System

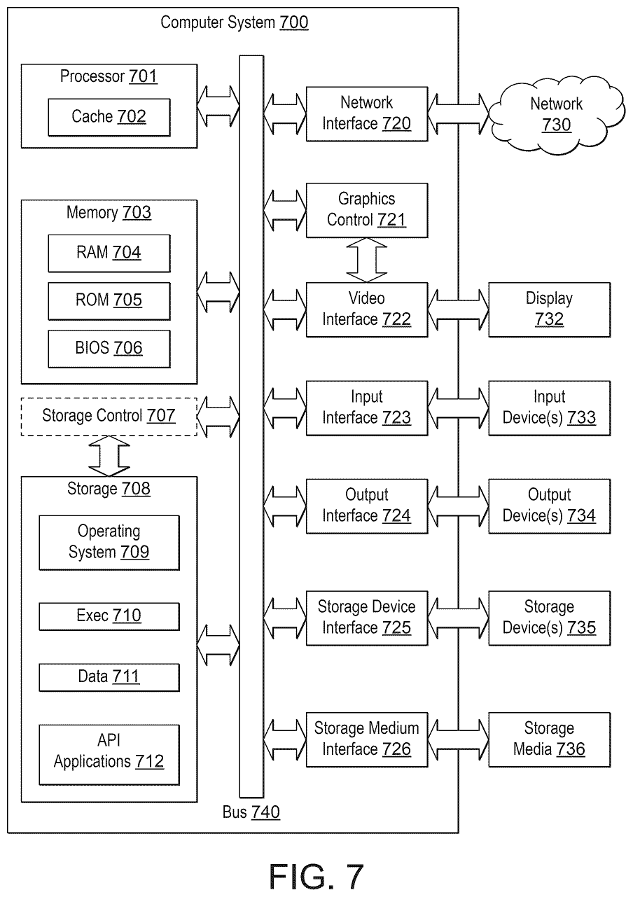

Referring to FIG. 7, a block diagram is shown depicting an exemplary machine that includes a computer system 700 (e.g., a processing or computing system) within which a set of instructions can execute for causing a device to perform or execute any one or more of the aspects and/or methodologies for static code scheduling of the present disclosure. The components in FIG. 7 are examples only and do not limit the scope of use or functionality of any hardware, software, embedded logic component, or a combination of two or more such components implementing particular embodiments.

Computer system 700 may include one or more processors 701, a memory 703, and a storage 708 that communicate with each other, and with other components, via a bus 740. The bus 740 may also link a display 732, one or more input devices 733 (which may, for example, include a keypad, a keyboard, a mouse, a stylus, etc.), one or more output devices 734, one or more storage devices 735, and various tangible storage media 736. All of these elements may interface directly or via one or more interfaces or adaptors to the bus 740. For instance, the various tangible storage media 736 can interface with the bus 740 via storage medium interface 726. Computer system 700 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 700 includes one or more processor(s) 701 (e.g., central processing units (CPUs) or general purpose graphics processing units (GPGPUs)) that carry out functions. Processor(s) 701 optionally contains a cache memory unit 702 for temporary local storage of instructions, data, or computer addresses. Processor(s) 701 are configured to assist in execution of computer readable instructions. Computer system 700 may provide functionality for the components depicted in FIG. 7 as a result of the processor(s) 701 executing non-transitory, processor-executable instructions embodied in one or more tangible computer-readable storage media, such as memory 703, storage 708, storage devices 735, and/or storage medium 736. The computer-readable media may store software that implements particular embodiments, and processor(s) 701 may execute the software. Memory 703 may read the software from one or more other computer-readable media (such as mass storage device(s) 735, 736) or from one or more other sources through a suitable interface, such as network interface 720. The software may cause processor(s) 701 to carry out one or more processes or one or more steps of one or more processes described or illustrated herein. Carrying out such processes or steps may include defining data structures stored in memory 703 and modifying the data structures as directed by the software.

The memory 703 may include various components (e.g., machine readable media) including, but not limited to, a random access memory component (e.g., RAM 704) (e.g., static RAM (SRAM), dynamic RAM (DRAM), ferroelectric random access memory (FRAM), phase-change random access memory (PRAM), etc.), a read-only memory component (e.g., ROM 705), and any combinations thereof. ROM 705 may act to communicate data and instructions unidirectionally to processor(s) 701, and RAM 704 may act to communicate data and instructions bidirectionally with processor(s) 701. ROM 705 and RAM 704 may include any suitable tangible computer-readable media described below. In one example, a basic input/output system 706 (BIOS), including basic routines that help to transfer information between elements within computer system 700, such as during start-up, may be stored in the memory 703.

Fixed storage 708 is connected bidirectionally to processor(s) 701, optionally through storage control unit 707. Fixed storage 708 provides additional data storage capacity and may also include any suitable tangible computer-readable media described herein. Storage 708 may be used to store operating system 709, executable(s) 710, data 711, applications 712 (application programs), and the like. Storage 708 can also include an optical disk drive, a solid-state memory device (e.g., flash-based systems), or a combination of any of the above. Information in storage 708 may, in appropriate cases, be incorporated as virtual memory in memory 703.

In one example, storage device(s) 735 may be removably interfaced with computer system 700 (e.g., via an external port connector (not shown)) via a storage device interface 725. Particularly, storage device(s) 735 and an associated machine-readable medium may provide non-volatile and/or volatile storage of machine-readable instructions, data structures, program modules, and/or other data for the computer system 700. In one example, software may reside, completely or partially, within a machine-readable medium on storage device(s) 735. In another example, software may reside, completely or partially, within processor(s) 701.

Bus 740 connects a wide variety of subsystems. Herein, reference to a bus may encompass one or more digital signal lines serving a common function, where appropriate. Bus 740 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 700 may also include an input device 733. In one example, a user of computer system 700 may enter commands and/or other information into computer system 700 via input device(s) 733. Examples of an input device(s) 733 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 733 may be interfaced to bus 740 via any of a variety of input interfaces 723 (e.g., input interface 723) including, but not limited to, serial, parallel, game port, USB, FIREWIRE, THUNDERBOLT, or any combination of the above.

In particular embodiments, when computer system 700 is connected to network 730, computer system 700 may communicate with other devices, specifically mobile devices and enterprise systems, distributed computing systems, cloud storage systems, cloud computing systems, and the like, connected to network 730. Communications to and from computer system 700 may be sent through network interface 720. For example, network interface 720 may receive incoming communications (such as requests or responses from other devices) in the form of one or more packets (such as Internet Protocol (IP) packets) from network 730, and computer system 700 may store the incoming communications in memory 703 for processing. Computer system 700 may similarly store outgoing communications (such as requests or responses to other devices) in the form of one or more packets in memory 703 and communicated to network 730 from network interface 720. Processor(s) 701 may access these communication packets stored in memory 703 for processing.

Examples of the network interface 720 include, but are not limited to, a network interface card, a modem, and any combination thereof. Examples of a network 730 or network segment 730 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 730, may employ a wired and/or a wireless mode of communication. In general, any network topology may be used.

Information and data can be displayed through a display 732. Examples of a display 732 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 732 can interface to the processor(s) 701, memory 703, and fixed storage 708, as well as other devices, such as input device(s) 733, via the bus 740. The display 732 is linked to the bus 740 via a video interface 722, and transport of data between the display 732 and the bus 740 can be controlled via the graphics control 721. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 732, computer system 700 may include one or more other peripheral output devices 734 including, but not limited to, an audio speaker, a printer, a storage device, and any combinations thereof. Such peripheral output devices may be connected to the bus 740 via an output interface 724. Examples of an output interface 724 include, but are not limited to, a serial port, a parallel connection, a USB port, a FIREWIRE port, a THUNDERBOLT port, and any combinations thereof.

In addition or as an alternative, computer system 700 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices may include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Select televisions, video players, and digital music players with optional computer network connectivity may be suitable for use in the system described herein. Suitable tablet computers, in various embodiments, may include those with booklet, slate, and convertible configurations.

In some embodiments, the computing device includes an operating system configured to perform executable instructions. The operating system is, for example, software, including programs and data, which manages the device's hardware and provides services for execution of applications. Suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Suitable media streaming device operating systems include, by way of non-limiting examples, Apple TV®, Roku®, Boxee®, Google TVR, Google Chromecast®, Amazon Fire®, and Samsung® HomeSync®. Suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, a web application, in various embodiments, can utilize one or more software frameworks and one or more database systems. In some embodiments, a web application is created upon a software framework such as Microsoft® NET or Ruby on Rails® (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. A web application, in various embodiments, may be written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript®, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

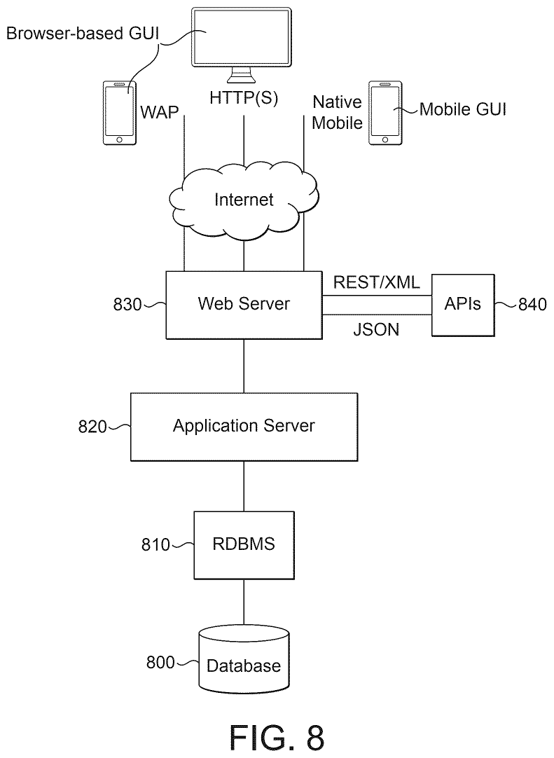

Referring to FIG. 8, in a particular embodiment, an application provision system comprises one or more databases 800 accessed by a relational database management system (RDBMS) 810. Suitable RDBMSs include Firebird, MySQL, PostgreSQL, SQLite, Oracle Database, Microsoft SQL Server, IBM DB2, IBM Informix, SAP Sybase, SAP Sybase, Teradata, MariaDB™ and the like. In this embodiment, the application provision system further comprises one or more application severs 820 (such as Java servers, .NET servers, PHP servers, and the like) and one or more web servers 830 (such as Apache, IIS, GWS and the like). The web server(s) optionally expose one or more web services via app application programming interfaces (APIs) 840. Via a network, such as the Internet, the system provides browser-based and/or mobile native user interfaces.

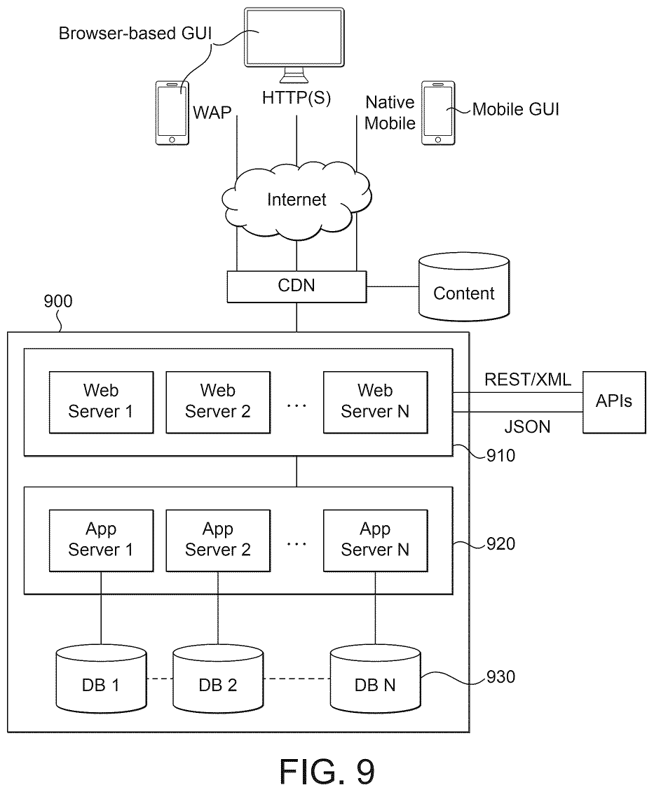

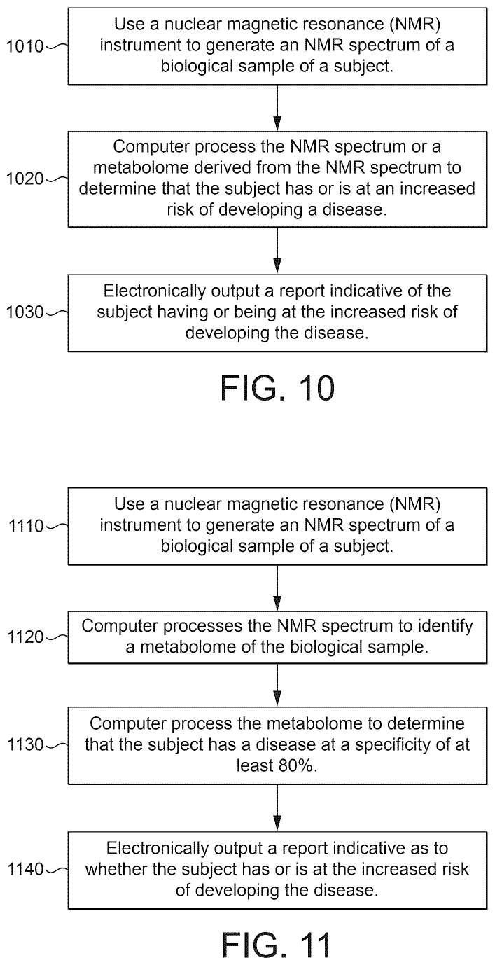

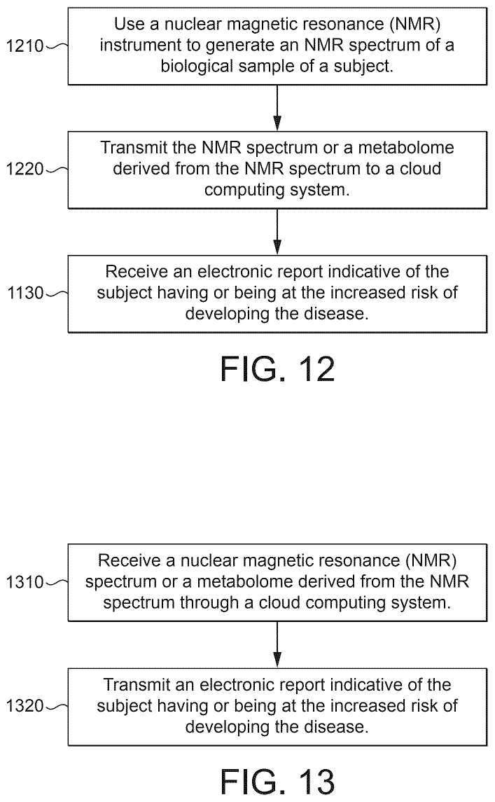

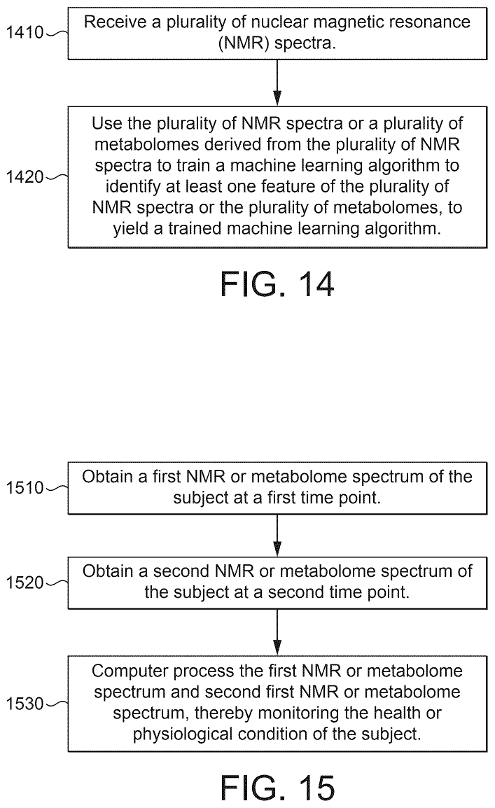

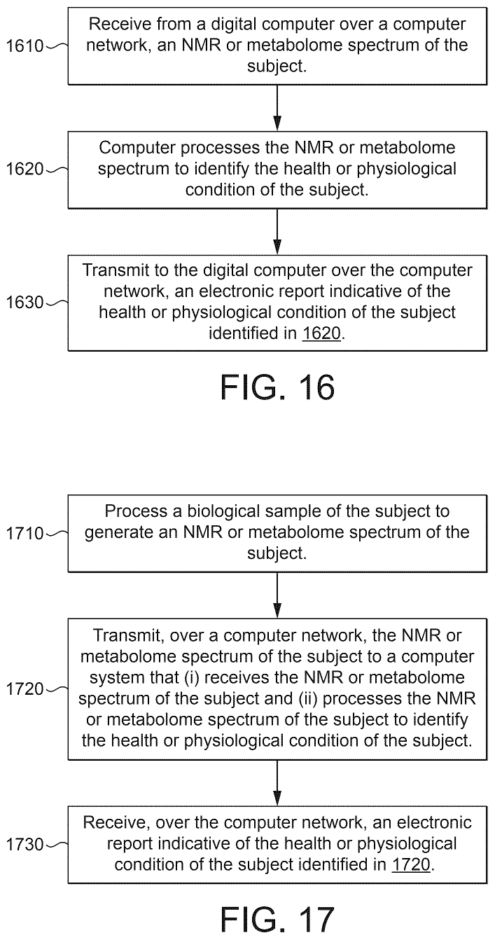

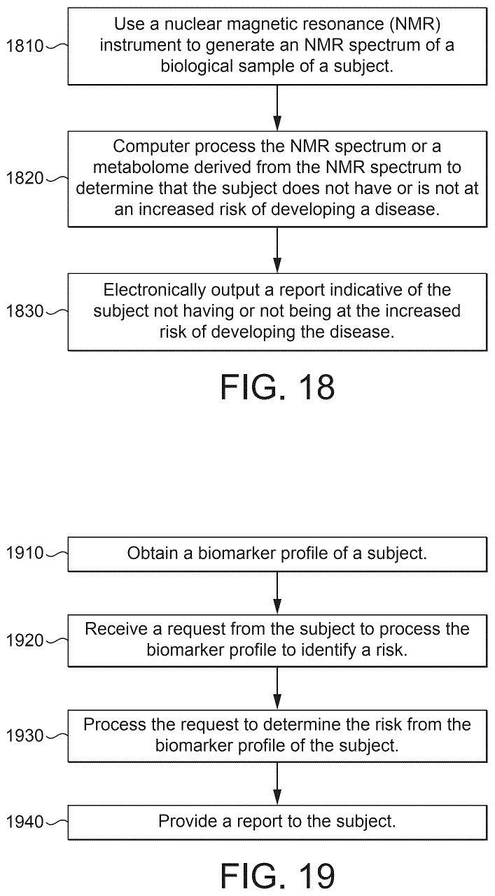

Referring to FIG. 9, in a particular embodiment, an application provision system alternatively has a distributed, cloud-based architecture 900 and comprises elastically load balanced, auto-scaling web server resources 910 and application server resources 920 as well synchronously replicated databases 930.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created using hardware, programming languages, and development environments known to the art. Mobile applications may be written in several languages. Suitable programming languages include, by way of non-limiting examples, C, C++, C#, Objective-C, Java™, Javascript®, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Standalone applications may be compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. There may exist several web browser plug-ins including, Adobe Flash Player, Microsoft Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google Android® browser, RIM BlackBerry® Browser, Apple Safari®, PalmX Blazer, Palm® WebOSX Browser, Mozilla Firefox for mobile, Microsoft Internet Explorer® Mobile, Amazon Kindle® Basic Web, Nokiax Browser, Opera Software” Opera Mobile, and Sony PSP™ browser.

### Software Modules

In some embodiments, the platforms, systems, media, and methods disclosed herein include software, server, and/or database modules, or use of the same. In view of the disclosure provided herein, software modules may be created using machines, software, and programming languages. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, many databases may be suitable for storage and retrieval data. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, MariaDB™, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Machine Learning

Many machine learning (ML) methods implemented as algorithms are suitable as approaches to perform the methods described herein. Such methods include but are not limited to supervised learning approaches, unsupervised learning approaches, semi-supervised approaches, or a combination thereof.

Machine learning algorithms may include without limitation neural networks (e.g., artificial neural networks (ANN), multi-layer perceptrons (MLP)), support vector machines, k-nearest neighbors, Gaussian mixture model, Gaussian, naive Bayes, decision trees, or radial basis functions (RBF). Linear machine learning algorithms may include without limitation linear regression, logistic regression, naive Bayes classifier, perceptron, or support vector machines (SVMs). Other machine learning algorithms for use with methods according to the disclosure may include without limitation quadratic classifiers, k-nearest neighbor, boosting, decision trees, random forests, neural networks, pattern recognition, Bayesian networks, or Hidden Markov models. Other machine learning algorithms, including improvements or combinations of any of these, commonly used for machine learning, can also be suitable for use with the methods described herein. Any use of a machine learning algorithm in a workflow can also be suitable for use with the methods described herein. The workflow can include, for example, cross-validation, nested-cross-validation, feature selection, row compression, data transformation, binning, normalization, standardization, and algorithm selection.

A machine learning algorithm can generally be trained by the following methodology:
1. Gather a dataset for “training” and “testing” the machine learning algorithm. The dataset can include many features, for example, concentrations of one or more metabolites, ratios of concentrations of two or more metabolites, or metadata described herein from a biological sample from a subject. The training dataset is used to “train” the machine learning algorithm. The testing dataset is used to “test” the machine learning algorithm.
2. Determine “features” for the machine learning algorithm to use for training and testing. The accuracy of the machine learning algorithm may depend on how the features are represented. For example, feature values may be transformed using one-hot encoding, binning, standardization, or normalization. Also, not all features in the dataset may be used to train and test the machine learning algorithm. Selection of features may depend on, for example, available computing resources and time or importance of features discovered during iterative testing and training. For example, it may be discovered that the feature of gender is important because this feature is predictive of a subject having or being at increased risk of developing a disease or disorder.
3. Choose an appropriate machine learning algorithm. For example, a machine learning algorithm described elsewhere herein may be chosen. The chosen machine learning algorithm may depend on, for example, available computing resources and time or whether the prediction is continuous or categorical in nature. The machine learning algorithm is used to build the machine learning model.
4. Build the machine learning model. The machine learning algorithm is run on the gathered training dataset. Parameters of the machine learning algorithm may be adjusted by optimizing performance on the testing dataset or via cross-validation datasets. After parameter adjustment and learning, the performance of the machine learning algorithm may be validated on a dataset of naive samples that are separate from the training dataset and testing dataset. The built machine learning model can involve feature coefficients, importance measures, or weightings assigned to individual features.

Once the machine learning model is determined as described above (“trained”), it can be used to make a prediction for a biological sample of a subject as having or being at increased risk of developing a disease or disorder. The prediction includes but is not limited to a classification of a biological sample as healthy or as having or being at increased risk of developing a disease or disorder.

### EXAMPLE

The following illustrative examples are representative of embodiments of the platform, software applications, systems, or methods described herein and are not meant to be limiting in any way.

### Example 1—Predicting Cancer Using Methods Described Herein

In an example, the methods disclosed herein were used in a clinical study to predict whether a subject had or was at risk of developing prostate cancer. The machine learning algorithm described herein was trained using a database comprising healthy subjects, subjects having prostate cancer, and subjects having other types of cancer. The database was used to generate, in part, a health book described elsewhere herein. Subjects included both male and female subjects shown in Table 1. Serum samples were collected from each subject in the study. Each sample was prepared and quantitatively measured using a Bruker 600 MHz NMR spectrometer equipped with a Double Resonance Broadband (BBI) probehead. The NMR raw data were digitized and processed to generate an NMR spectrum for each subject. The NMR spectrum of each subject was then quantitatively translated into concentration levels of individual substances to generate a metabolome for each subject. Each metabolome was labelled according to each diagnosis shown in Table 1. The machine learning algorithm was then trained using the labeled metabolomes from each subject in the study. The self-reported healthy subjects served as a control group for the machine learning algorithm. The machine learning algorithm learned to distinguish healthy control subjects from other subjects having prostate cancer using a biomarker panel of 9 concentrations of metabolites or ratios of concentrations of metabolites. Further, the machine learning algorithm learned to distinguish between the six named cancers of colon, rectal, pancreatic, breast, ovarian, and prostate cancer as well as nonspecific cancer and generally sick. The biomarker panel functioned as a disease specific signature for prostate cancer. The biomarker panel showed significant deviations in concentrations of metabolites or ratios of concentrations of metabolites compared to healthy subject controls. For example, the machine learning algorithm was able to predict a subject having prostate cancer or at risk of having prostate cancer with an area under curve (AUC) of about 0.986.

TABLE 1

| | Mean | Total | Male | Female | Sample |
|---|---|---|---|---|---|
| Diagnosis | Age | Subjects | Subjects | Subjects | Type |
| Self-reported healthy | 47 | 775 | 494 | 281 | Serum |
| Sick | 61 | 1366 | 676 | 690 | Serum |
| Cancer nonspecific | 68 | 523 | 292 | 231 | Serum |
| Colon cancer | 76 | 58 | 27 | 31 | Serum |
| Rectal cancer | 70 | 61 | 39 | 22 | Serum |
| Pancreatic cancer | 66 | 51 | 36 | 15 | Serum |
| Breast cancer | 61 | 94 | 0 | 94 | Serum |
| Ovarian cancer | 61 | 69 | 0 | 69 | Serum |
| Prostate cancer | 73 | 190 | 190 | 0 | Serum |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-294. (canceled)

295. A method for processing a biological sample of a subject, comprising:
(a) using a nuclear magnetic resonance (NMR) instrument to generate an NMR spectrum of the biological sample;
(b) computer processing the NMR spectrum or a metabolome derived from the NMR spectrum to determine that the subject has or is at an increased risk of developing a disease, wherein the computer processing comprises use of a database comprising at least about 100,000 NMR spectra; and
(c) electronically outputting a report indicative of the subject having or being at the increased risk of developing the disease.

296. The method of claim 295, wherein said disease is a cancer.

297. The method of claim 296, wherein said cancer comprises breast cancer, prostate cancer, or lung cancer.

298. The method of claim 295, wherein said disease is a metabolic disease.

299. The method of claim 298, wherein said metabolic disease comprises diabetes or non-alcoholic fatty liver disease.

300. The method of claim 298, wherein said disease is an inflammatory disease.

301. The method of claim 300, wherein said inflammatory disease comprises rheumatoid arthritis or ulcerative colitis.

302. The method of claim 295, wherein said computer processing comprises use of a trained machine learning algorithm.

303. The method of claim 302, wherein said trained machine learning algorithm comprises use of one or more artificial neural networks.

304. The method of claim 295, wherein said biological sample comprises bile acids, blood, bronchoalveolar lavage fluid, cell extracts, cerebrospinal fluid, exhaled breath condensates (EBC), plasma, saliva, serum, sperm, stool, sweat, tear fluid, tracheal wash (TW), or urine.

305. The method of claim 295, wherein said metabolome comprises information related to at least about 100 metabolites.

306. The method of claim 295, wherein said report has an accuracy, selectivity, or specificity of at least about 80%.

307. A system for processing or analyzing a biological sample of a subject, comprising:
one or more computer processors that are individually or collectively programmed to:
(i) process a nuclear magnetic resonance (NMR) spectrum of said biological sample of said subject, or a metabolome derived from said NMR spectrum, to determine that said subject has or is at an increased risk of developing a disease, wherein said NMR spectrum or said metabolome corresponds to a plurality of metabolites; and
(ii) output a report indicative of said subject having or being at said increased risk of developing said disease.

308. The system of claim 307, further comprising computer memory operatively coupled to said one or more computer processors, wherein said computer memory stores said NMR spectrum or said metabolome.

309. The system of claim 307, wherein said one or more computer processors are within a cloud computing system.

310. A method for processing or analyzing a biological sample of a subject, comprising:
(a) receiving from a digital computer over a computer network, an NMR or metabolome spectrum of the subject;
(b) computer processing the NMR or metabolome spectrum to identify the health or physiological condition of the subject; and
(c) transmitting to the digital computer over the computer network, an electronic report indicative of the health or physiological condition of the subject identified in (b).

311. The method of claim 310, wherein said computer processing comprises determining a deviation of said NMR spectrum or said metabolome from a healthy set of information stored in said database.

312. The method of claim 310, wherein a time from (a) to (c) is less than about 60 minutes.

313. The method of claim 310, wherein said biological sample is not destroyed.

314. The method of claim 310, wherein said computer processing comprises deconvoluting said spectrum or said metabolome to determine a presence or absence of said plurality of metabolites.
